(19) United States
(12) Patent Application Publication
(10) Pub. No.: US 2020/0254429 A1
(43) Pub. Date: Aug. 13, 2020

(54) PROCESS AND CATALYSTS FOR THE OXIDATION AND/OR AMMOXIDATION OF OLEFIN

(71) Applicants: King Abdullah University of Science and Technology, Thuwal (SA); CENTRE NATIONAL DE LA RECHERCHE SCIENTIFIQUE, Paris (FR); UNIVERSITY CLAUDE BERNARD LYON 1, VILLEUBANNE (FR); ECOLE SUPERIEURE DE CHIMIE PHYSIQUE ELECTRONIQUE DE LYON, VILLEUBANNE (FR)

(72) Inventors: Samir BARMAN, Thuwal (SA); Manoja Kumar SAMANTARAY, Thuwal (SA); Youssef SAIH, Thuwal (SA); Jean Marie BASSET, Thuwal (SA); Edy ABOU HAMMAD, Thuwal (SA); Mostafa TAOUFIK, Paris (FR); Nicolas MERLE, Paris (FR); Kai Chung SZETO, Paris (FR); Frederic LE QUEMENER, Paris (FR); Aimery DE MALLMANN, Paris (FR)

(21) Appl. No.: 16/637,522

(22) PCT Filed: Aug. 7, 2018
(86) PCT No.: PCT/IB2018/055956

§ 371 Date: Feb. 7, 2020

Related U.S. Application Data
(60) Provisional application No. 62/541,973, filed on Aug. 7, 2017.
(60) Provisional application No. 62/645,851, filed on Mar. 21, 2018.

(51) Int. Cl. B01J 31/16; B01J 23/18; B01J 21/08; B01J 23/28; B01J 31/22; B01J 31/18; B01J 31/12; B01J 37/02; C07C 45/35; C07C 45/34; C07C 253/26

(52) U.S. Cl. CPC B01J 31/1633; B01J 2231/321; B01J 21/08; B01J 23/28; B01J 31/2239; B01J 31/1805; B01J 31/122; B01J 31/2286; B01J 37/0209; B01J 37/0207; C07C 45/35; C07C 45/34; C07C 253/26; B01J 2531/0216; B01J 2531/64; B01J 2531/54; B01J 23/18

(57) ABSTRACT

Embodiments of the present disclosure describe a catalyst and/or a precatalyst, in particular a single site catalyst and/or a single site precatalyst, for the oxidation and/or ammoxidation of olefins to produce aldehydes and/or nitriles, methods of preparing a corresponding catalyst and/or precatalyst, in particular single site catalyst and/or single site precatalyst, and methods of using said catalyst and/or precatalyst, in particular said single site catalyst and/or single site precatalyst, to produce aldehydes and/or nitriles.

### BACKGROUND

Acrylonitrile is an important organic chemical raw material, which is historically produced by the ammoxidation of propylene. Acrylonitrile is in particular a very important monomer for the manufacture of useful plastics. While most of acrylonitrile's major end markets (e.g., polyacrylonitrile, acrylonitrile-butadiene-styrene (ABS) resins, acrylic fiber, and adiponitrile) are cyclical and impacted by economic downturns, it remains a key chemical intermediate of high demand, which is expected to grow through at least 2021. New demand for acrylonitrile and its derivatives now comes from the emerging world, where improvements in living standards drive demand for products such as appliances, automobiles, electronics, textiles, clothing, consumables, and the development of infrastructure. Production of ABS is the primary end use for acrylonitrile globally, accounting for about a third of the overall demand. Its most common production process—known since the nineteen fifties—is called the SOHIO process (from the Standard Oil Company, Ohio), i.e. the catalytic ammoxidation of propylene in the presence of ammonia and air. For example, U.S. Pat. No. 2,904,580 claimed a process for the manufacture of acrylonitrile comprising the step of contacting in the vapor phase a mixture of propylene, ammonia and oxygen with a bismuth phosphomolybdate catalyst. More recently, US2004106817 disclosed a process for the conversion of propylene to acrylonitrile by reacting in the vapor phase at an elevated temperature and pressure said propylene with a molecular oxygen containing gas and ammonia in the presence of an ammoxidation catalysts which comprise rubidium, cerium, chromium, magnesium, iron, bismuth, molybdenum, and at least one of nickel or nickel and cobalt, in the substantial absence of manganese, a noble metal or vanadium.

The production of acetonitrile was, for example, disclosed in U.S. Pat. No. 2,432,532, which was granted to Philips Petroleum Company. The patent disclosed that acetonitrile could be efficiently and economically produced from the interaction of olefins with three to five carbon atoms per molecule and ammonia over a contact catalyst comprising natural or synthetic alumina. Today, acetonitrile is essentially obtained as a by-product from the manufacture of acrylonitrile.

Ethanal (acetaldehyde) is one of the most important aldehydes, occurring widely in nature and being produced on a large scale in industry. It is mainly used as a building block for various important products, such as pyridine derivatives, vinyl acetate, pentaerythritol, crotonaldehyde, and resin. Ethylene is the dominant feedstock for the production of ethanal. The stoichiometric ethylene oxidation reaction has been discovered by F. C. Phillips in 1894. It is back in the early nineteen fifties that the industrial production process of acetaldehyde from ethylene was developed. It is known as the Wacker process, which involves oxidation of ethylene using a homogeneous palladium/copper system, and it is still an improvement of the same Wacker process, which is in use today for the production of millions of tonnes of acetaldehyde on an annual basis. For example, U.S. Pat. No. 3,076,032 discloses a process for the conversion of an olefinic hydrocarbon having 2 to 4 carbon atoms to e.g. acetaldehyde, in the presence of oxygen, water and a catalyst of (a) palladium chloride and (b) as a redox system, copper chloride. This industrial process comprises several drawbacks. The homogeneously catalysed Wacker process requires several distillation towers in order to achieve the proper purity. Moreover, generation of the highly corrosive HCl upon the catalytic reaction also imposes the use of devoted materials (normally ceramic coated titanium) for the construction of the reactor and the plant. Furthermore, the homogeneous nature of the process complicates the regeneration of the catalysts.

Propenal (acrolein) is the simplest unsaturated aldehyde which is produced industrially from propylene and mainly used as a biocide and a building block to other chemical compounds. In 1948, Shell developed the first industrial process for the selective oxidation of propene to acrolein using a supported copper oxide catalyst. For example, U.S. Pat. No. 2,451,485 claims a process for the production of acrolein by passing a gaseous mixture comprising propene and oxygen into contact with a solid catalyst based on cuprous oxide. Subsequently, Standard Oil of Ohio (Sohio) discovered that catalysts based on bismuth molybdates had excellent selectivity for this reaction. Today, acrolein is exclusively produced by this selective oxidation of propene route using catalysts composed of different transition metals comprised in complex metal oxides.

There is thus still a demand for the industrial production of acrylonitrile, acetaldehyde, acetonitrile and/or acrolein from olefins in a process which is efficient and commercially valuable.

### SUMMARY

In general, embodiments of the present disclosure describe a catalyst and/or a precatalyst, in particular a single site catalyst and/or a single site precatalyst, for the oxidation and/or ammoxidation of olefins to produce aldehydes and/or nitriles, methods of preparing a corresponding catalyst and/or precatalyst, in particular single site catalyst and/or single site precatalyst, and methods of using said catalyst and/or precatalyst, in particular said single site catalyst and/or single site precatalyst, to produce aldehydes and/or nitriles.

Accordingly, embodiments of the present disclosure describe a catalyst and/or precatalyst comprising a support and an inorganic and/or organometallic complex grafted on the support. In an embodiment, the support includes one or more of inorganic oxide, silicon-modified inorganic oxide, and bismuth-modified inorganic oxide. In an embodiment, the inorganic and/or organometallic complex includes one or more of Group V elements, Group VI elements, and Group VII elements.

Embodiments of the present disclosure describe a catalyst and/or a precatalyst, in particular a single site catalyst and/or a single site precatalyst, exhibiting high selectivity towards aldehydes and/or nitriles with a defined and low load active metal catalyst and/or precatalyst specie(s).

Embodiments of the present disclosure further describe a method of preparing a catalyst and/or a precatalyst, in particular a single site catalyst and/or a single site precatalyst comprising the grafting of inorganic and/or organometallic complexes of group V elements (Ta, V and Nb), group VI elements (preferably Mo, W and/or Cr) or Rhenium (Re) or Rhodium (Rh) or Ruthenium (Ru) on inorganic oxide and/or on bismuth modified inorganic oxide.

Embodiments of the present disclosure describe a method of making a catalyst and/or precatalyst comprising treating one or more of an inorganic oxide support, silicon-modified inorganic oxide support, and bismuth-modified inorganic oxide support at or to a temperature ranging from about 100° C. to about 900° C.; and grafting one or more of an inorganic and/or organometallic complex on the support.

Another embodiment of the present disclosure is a method of using a catalyst and/or a precatalyst, in particular a single site catalyst and/or a single site precatalyst, comprising contacting an olefin with the said catalyst and/or precatalyst, in particular the said single site catalyst and/or single site precatalyst, in the presence of oxygen and optionally ammonia to produce aldehydes and/or nitriles.

Another embodiment of the present disclosure is a process for manufacturing aldehydes/nitriles from an olefin which comprises the reaction of an olefin in the presence of oxygen, optionally ammonia, and a catalyst and/or precatalyst as structurally defined herein below, in particular a single site catalyst and/or single site precatalyst.

Still another embodiment of the present disclosure is a process for manufacturing aldehydes/nitriles from an olefin which comprises the reaction of an olefin in the presence of oxygen, optionally ammonia, and a catalyst (and/or a precatalyst) comprising monomeric or dimeric inorganic and/or organometallic complexes of group V elements (Ta, V and Nb), group VI elements (preferably Mo, W and/or Cr), group VII elements (preferably Rhenium), group VIII elements (e.g., Ruthenium (Ru)), and/or group IX elements (e.g., Rhodium (Rh)) grafted on inorganic oxide and/or on silicon or bismuth modified inorganic oxide.

Another embodiment of the present disclosure is a method of making one or more of aldehydes and nitriles comprising contacting an olefin and one or more of oxygen and ammonia in a presence of a catalyst to produce one or more of aldehydes and nitriles. In an embodiment, the catalyst is a single-site catalyst including an inorganic and/or organometallic complex grafted on a support.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

Reference is made to illustrative embodiments that are depicted in the figures, in which:

FIG. 1 depicts the selectivity vs time on stream plot of propene oxidation under select conditions over (≡SiO)₂Mo(═N-t-Bu)₂ (reaction conditions: T=380° C.; Feed ratio C₃═/NH₃/O₂/N₂/He=7.5/0/10/12.5/70), according to one or more embodiments of the present disclosure.

FIG. 2 depicts the selectivity vs time on stream plot of propene ammoxidation under selected condition over (≡SiO)₂Mo(═N-t-Bu)₂ (reaction conditions: T=380° C.; Feed ratio: C₃═/NH₃/O₂/N₂/He=7.5/7.5/7.5/12.5/65), according to one or more embodiments of the present disclosure.

FIG. 3 depicts the selectivity vs time on stream plot of propene oxidation under selected condition over MoO₂Mes₂/SiO₂₋₂₀₀ (reaction conditions: T=380° C.; Feed ratio: C₃═/NH₃/O₂/N₂/He=7.5/0/10/12.5/70), according to one or more embodiments of the present disclosure.

FIG. 4 depicts the selectivity vs time on stream plot of propene ammoxidation under selected condition over MoO₂Mes₂/SiO₂₋₂₀₀ (Feed ratio: C₃═/NH₃/O₂/N₂/He=7.517.5/10/12.5/62.5), according to one or more embodiments of the present disclosure.

FIG. 5 depicts the selectivity vs time on stream plot of propene oxidation under selected condition over Mo(═N-(2,6-C₆H₃-ⁱPr₂)₂(CH₂tBu)₂/Bi₂O₃—SiO₂₋₂₀₀ (feed ratio: C₃═/NH₃/O₂/N₂/He=7.5/0/10/12.5/70), according to one or more embodiments of the present disclosure.

FIG. 6 depicts the selectivity vs time on stream plot of propene oxidation (FIG. 5) and ammoxidation (FIG. 6) under selected condition over Mo(═N-(2,6-C₆H₃-ⁱPr₂)₂(CH₂tBu)₂/Bi₂O₃—SiO₂₋₂₀₀ (feed ratio: C₃═/NH₃/O₂/N₂/He=7.5/7.5/2.5/12.5/70), according to one or more embodiments of the present disclosure.

FIG. 7 depicts the selectivity vs time on stream plot of propene oxidation under selected condition over Mo(═N-t-Bu)₂(CH₂tBu)₂/SO₂—Bi₂O₃₋₍₂₀₀₎ (Feed ratio C₃═/NH₃/O₂/N₂/He=7.5/0/10/12.5/70), according to one or more embodiments of the present disclosure.

FIG. 8 depicts the selectivity vs time on stream plot of propene oxidation ammoxidation under selected condition over Mo(═N-t-Bu)₂(CH₂tBu)₂/SiO₂—Bi₂O₃₋₍₂₀₀₎ (Feed ratio: C₃═/NH₃/O₂/N₂/He=7.5/7.5/2.5/12.5/70), according to one or more embodiments of the present disclosure.

FIG. 9 depicts the selectivity vs time on stream plot of propene oxidation under selected condition over Mo(═O)(CH₂^{t}Bu)₃Cl/SO₂—Bi₂O₃₋₍₂₀₀₎ catalyst (Reaction conditions: T=380° C.; weight of catalyst: 36 mg; Feed ratio: C₃═/NH₃/O₂/N₂/He=7.5/0/10/12.5/70), according to one or more embodiments of the present disclosure.

FIG. 10 depicts the selectivity vs time on stream plot of propene ammoxidation under selected condition over Mo(═O)(CH₂^{t}Bu)₃Cl/SO₂—Bi₂O₃₋₍₂₀₀₎ catalyst (Feed ratio: C₃═/NH₃/O₂/N₂/He=7.517.5/10/12.5/62.5), according to one or more embodiments of the present disclosure.

FIG. 11 depicts the selectivity vs time on stream plot of propene oxidation over Mo(≡C^{t}Bu)(CH₂^{t}Bu)₃/Bi₂O₃—SiO₂₋₂₀₀ (Feed ratio: C₃═/NH₃/O₂/N₂/He=7.5/0/5/12.5/75), according to one or more embodiments of the present disclosure.

FIG. 12 depicts the selectivity vs time on stream plot of propene ammoxidation over Mo(≡C^{t}Bu)(CH₂^{t}Bu)₃/Bi₂O₃—SiO₂₋₂₀₀ (Feed ratio: C₃═/NH₃/O₂/N₂/He=7.5/7.5/5/12.5/67.5), according to one or more embodiments of the present disclosure.

FIGS. 13A-13D show a comparison of the catalytic cycles for the oxidation of olefins to aldehydes and the reaction mechanisms: a) Bimetallic Wackercycle for the oxidation of ethylene to acetaldehyde; b) Cross metathesis of ethylene and 2-butene to propylene; c) Metathetic-oxidation of 2-butene and molecular oxygen to acetaldehyde; d) Cycle based on a silica-supported Mo (bis-oxo) single-atom species ((≡Si—O—)₂Mo(═O)₂) for the metathetic-oxidation of 2-butene to acetaldehyde, according to one or more embodiments of the present disclosure.

FIG. 14 is a schematic illustration of the preparation steps for the single-site (≡Si—O—)₂Mo(═O)₂ catalyst, according to one or more embodiments of the present disclosure.

FIGS. 15A-15C are graphical views of (A) DRIFT spectra of silica dehydroxylated at 200° C. (black), (≡Si—O—)₂Mo(═O)O^{t}Bu)₂ (red), and (≡Si—O—)₂Mo(═O)₂ (blue); B,C) Solid-state (B) ¹HNMR and (C) ¹³C NMR spectra of (≡Si—O—)₂Mo(═O)(O^{t}Bu)₂, according to one or more embodiments of the present disclosure.

FIG. 16 is a schematic diagram of a proposed mechanism for the formation of (≡Si—O—)₂Mo(═O)₂ from (—SI—O—)₂Mo(═O)(O^{t}Bu)₂ through a combination of β-H elimination and alcohol condensation, according to one or more embodiments of the present disclosure.

FIG. 17 is a graphical view of Raman spectra of Mo bis-oxo species (≡Si—O—)₂Mo(═O)₂, according to one or more embodiments of the present disclosure.

FIG. 18 is a graphical view of DR UV-Vis spectrum of (≡Si—O—)₂Mo(═O)(O^{t}Bu)₂, according to one or more embodiments of the present disclosure.

FIGS. 19A-19C are graphical views of (A) Molybdenum (Mo) K-edge normalized absorption spectra of (≡Si—O—)₂Mo(═O)₂ (line A, red) and a metallic Mo foil (line B, blue): (B) Mo K-edge k³-weighted EXAFS for (≡Si—O—)₂Mo(═O)₂, resulting from the grafting reaction of Mo(═O)(O^{t}Bu)₄ onto SiO₂₋₂₀₀ followed by thermal treatment at 250° C.; (C) Corresponding Fourier transform (modulus and imaginary part); solid lines: experimental; dashed lines: fit, according to one or more embodiments of the present disclosure.

FIGS. 20A-20B are graphical views of a plot (left) at 400° C. and (right) at 450° C., represent conversion and selectivity vs time-on-stream for propylene oxidation over (≡Si—O—)₂Mo(═O)₂ (Reaction conditions: Cat. wt: 150 mg; Feed ratio: C₃═/O₂/N₂/He=7.5/10/12.5/70; Total flow: 32 mL/min; Gas hourly space velocity (GHSV) of 12800 h⁻¹; W/F: 0.281 g·s·mL⁻¹), according to one or more embodiments of the present disclosure.

FIG. 21 is a graphical view showing conversion and selectivity vs time-on-stream plot of propylene oxidation over (≡Si—O—)₂Mo(═O)₂ at 450° C. over a prolonged experimental time of 16 h (Reaction conditions: Cat. wt: 150 mg; Feed ratio: C₃═/O₂/N₂/He=7.5/10/12.5/70; Total flow: 32 mL/min; Gas hourly space velocity (GHSV) of 12800 h⁻¹; W/F: 0.281 g·s·mL⁻¹), according to one or more embodiments of the present disclosure.

FIGS. 22A-22B are graphical views showing the conversion and selectivity vs time-on-stream plot of propylene oxidation at 450° C. over catalyst, (≡Si—O—)₂Mo(═O)₂ (Reaction conditions: Cat. wt: 150 mg; Feed ratio: C₃═/O₂/N₂/He=7.5/5/12.5/75 (for left); Feed ratio: C₃═/O₂/N₂/He=7.5/2.5/12.5/77.5 (for right); Total flow: 32 mL/min; Gas hourly space velocity (GHSV) of 12800 h⁻¹; W/F: 0.281 g·s·mL⁻¹), according to one or more embodiments of the present disclosure.

FIG. 23 is a schematic diagram of a proposed mechanistic pathway for the formation of formaldehyde from α-olefins, according to one or more embodiments of the present disclosure.

FIGS. 24A-24B are graphical views of conversion and selectivity as a function of time-on-stream for the cis-2-butene oxidation over (≡Si—O—)₂Mo(═O)₂ at 350° C. (left) and at 400° C. (right) (Reaction conditions: Cat. wt: 200 mg; Feed ratio: C₃═/O₂/N₂/He=7.5/10/12.5/70; Total flow: 32 mL/min; Gas hourly space velocity (GHSV) of 9600 h⁻¹; W/F: 0.375 g·s·mL⁻¹ (other products with inferior selectivity were omitted for clarity), according to one or more embodiments of the present disclosure.

FIG. 25 is a graphical view showing the conversion and selectivity vs time-on-stream plot of cis-2-butene oxidation over (≡Si—O—)₂Mo(═O)₂ at 450° C. (Reaction conditions: Cat. wt: 200 mg; Feed ratio: C₄═/O₂/N₂/He=7.5/10/12.5/70; Total flow: 32 mL/min; Gas hourly space velocity (GHSV) of 9600 h⁻¹; W/F: 0.375 g·s·mL⁻¹), according to one or more embodiments of the present disclosure.

FIG. 26 is a graphical view showing the conversion and selectivity vs time-on-stream plot of cis-2-butene oxidation over (≡Si—O—)₂Mo(═O)₂ at 400° C. over a prolong experimental time of about 20 h (Reaction conditions: Cat. wt: 200 mg; Feed ratio: C₄═/O₂/N₂/He=7.5/10/12.5/70; Total flow: 32 mL/min; Gas hourly space velocity (GHSV) of 9600 h⁻¹; W/F: 0.375 g·s·mL⁻¹), according to one or more embodiments of the present disclosure.

FIG. 27 is a graphical view showing the conversion and selectivity vs time-on-stream plot of cis-2-pentene oxidation over (≡Si—O—)₂Mo(═O)₂ at 400° C. (Reaction conditions: Cat. wt: 75 mg; Feed ratio: C₅═/O₂/N₂/He=5.9/7.8/12.5/73.8; Total flow: 16 mL/min; Gas hourly space velocity (GHSV) of 12800 h⁻¹; W/F: 0.281 g·s·mL⁻¹), according to one or more embodiments of the present disclosure.

FIGS. 28A-28C are schematic diagrams of (A) reaction pathway for oxidation of cis-2-butene to acetaldehyde by O₂ and catalyzed by a model of (≡Si—O—)₂Mo(═O)₂. The DFT-calculated ΔG (kcal/mol) values are reported in blue near the structure labels; (B) Geometry of transition state TS2 with selected distances in Å; (C) Geometry of transition state TS3 with selected distances in A, according to one or more embodiments of the present disclosure.

FIG. 29 is a schematic diagram of the silica cluster model used in the present work. Color coding: Si (gray), O (red), H (gray), according to one or more embodiments of the present disclosure.

FIG. 30 is an intrinsic reaction coordinate plot showing the connection between transition state TS2, presenting a large C—C distance of 2.86 Å, and the preceding intermediate II, according to one or more embodiments of the present disclosure.

### DETAILED DESCRIPTION

The invention of the present disclosure relates to novel catalyst and/or precatalyst, in particular novel single site catalyst and/or single site precatalyst. In particular, the invention of the present disclosure relates to a catalyst and/or a precatalyst, in particular a single site catalyst and/or a single site precatalyst, methods of preparing a catalyst and/or a precatalyst, in particular a single site catalyst and/or a single site precatalyst, and methods of using a catalyst and/or a precatalyst, in particular a single site catalyst and/or a single site precatalyst. The catalyst and/or a precatalyst, in particular the single site catalyst and/or single site precatalyst, of the present disclosure may be contacted with olefin(s) in the presence of oxygen and optionally ammonia to produce aldehydes and/or nitriles.

The present invention differs from the prior art of ammoxidation to produce acrylonitrile and/or acetonitrile in that the present invention provides a novel olefin ammoxidation reaction performed in the presence of a catalyst and/or a precatalyst, in particular a single site catalyst and/or precatalyst, exhibiting high selectivity with a defined and low load active metal catalyst specie. Indeed, the present invention differs from traditional heterogeneous catalysis methods by the controlled introduction of inorganic and/or organometallic molecular complexes onto well-defined supports and by the presence of low concentration of active metal on the surface of said supports compared to classical heterogeneous catalysis applied for those reactions.

The present invention differs from the prior art of oxidation to produce ethanal and/or propenal in that the present invention provides a novel olefin oxidation reaction performed in the presence of a catalyst and/or a precatalyst, in particular a single site catalyst and/or single site precatalyst, exhibiting high selectivity with a well-defined and low load supported active metal catalyst specie. Indeed, the present invention differs from traditional heterogeneous catalysis methods by the controlled introduction of inorganic and/or organometallic molecular complexes onto well-defined inorganic supports.

At least one benefit of the present invention is that our claimed single site catalyst and/or a single site precatalyst will enable the use of continuous flow reactor that will first of all simplify the production plant and increase the economic benefit with respect to the existing Wacker process using the homogeneous CuCl₂/PdCl₂ catalysts; additionally, the catalysts can easily be regenerated. Moreover, the approach of metathesis between an olefin and molecular oxygen will offer a green process for the preparation of various carbonyl compounds in both academia and industry.

### Definitions

The terms recited below have been defined as described below. All other terms and phrases in this disclosure shall be construed according to their ordinary meaning as understood by one of skill in the art.

The Periodic Table of the Elements mentioned in this description is that drawn up by the IUPAC in 1991, in which the groups are numbered from 1 to 18. It is found, for instance, in “CRC Handbook of Chemistry and Physics” 76^{th} Edition (1995-1996) by David R. Lide and published by CRC Press, Inc. (USA).

As used herein, “contacting” refers to the act of touching, making contact, or of bringing to immediate or close proximity, including at the cellular or molecular level, for example, to bring about a physiological reaction, a chemical reaction, or a physical change.

As used herein, “dehydrating” and “dehydration” refers to reducing a content of water (e.g., water as a vapor, gas, solid, etc.).

As used herein, “single site catalyst” refers to a catalyst in which more than 80% (for example as measured by elemental analysis and/or solid state NMR and/or confirmed by XAFS spectroscopy) of the sites are structurally identical, preferably more than 90%, more preferably more than 95%, or even all of the sites are structurally identical. The metal of these single site catalysts can advantageously be selected from group V (Ta, Nb, V), group VI (Mo, W, Cr), group VII (Rhenium), group VIII (Ruthenium), group IX (Rhodium), preferably from group VI (Mo, W, Cr) and Rhenium. Examples of single site catalysts or precatalysts can be found e.g. in Pelletier and Basset Acc. Chem. Res., 2016, 49 (4), pp 664-677). Illustrative examples of analysis of the single site catalyst (or precatalyst) of the present invention can be found in Y Bouhoute et al. ACS Catal., 2016, 6 (1), pp 1-18 (“Accessing Realistic Models for the WO₃—SiO₂ Industrial Catalyst through the Design of Organometallic Precursors”) and/or in N Merle et al. J Am Chem Soc 2017, 139 (6), 2144-2147 (“Well-Defined Molybdenum Oxo Alkyl Complex Supported on Silica by Surface Organometallic Chemistry: A Highly Active Olefin Metathesis Precatalyst”); the structure of resulting species were e.g. determined by DRIFT, RAMAN, elemental analysis, Solid state NMR and XAFS.

As used herein, “precatalyst” is a chemical specie which converts (e.g. when used in the olefin conversion process of the present invention) into a corresponding active catalyst.

As used herein, “single site precatalyst” is a chemical specie which converts (e.g. when used in the olefin conversion process of the present invention) into a corresponding active single site catalyst.

The Applicants have surprisingly discovered a supported metal compound capable of acting as catalyst and improving catalytic reactions of saturated or unsaturated hydrocarbons. The improvements can be preferably observed in the oxidation and/or ammoxidation of olefins to produce aldehydes and/or nitriles, and more particularly conversion of propylene into ethanal and/or propenal and/or acrylonitrile and/or acetonitrile. More particularly, these reactions can be performed with an enhanced selectivity towards the aldehydes/nitriles (e.g. ethanal and/or propenal and/or acrylonitrile and/or acetonitrile). These results can be obtained in catalytic reactions involving alkenes, e.g. internal olefins or alpha-olefins, particularly linear alkenes (especially propylene) or branched alkenes e.g. having an “iso” structure, e.g. isobutene.

### Catalyst

In an embodiment, the present invention relates to a single site catalyst and/or single site precatalyst comprising a support and an inorganic and/or organometallic complex grafted on the support.

The support may include any of the supports of the present disclosure. For example, in an embodiment, the support may include one or more of inorganic oxide, silicon-modified inorganic oxide, and bismuth-modified inorganic oxide. In an embodiment, the support includes one or more of silica, bismuth oxide, bismuth-modified silica, and silicon-modified bismuth oxide.

The inorganic and/or organometallic complex may include any of the metals and/or metal complexes of the present disclosure. In an embodiment, the inorganic and/or organometallic complex may include one or more of Group V elements, Group VI elements, and Group VII elements. For example, in an embodiment, the inorganic and/or organometallic complex may include one or more of vanadium, niobium, tantalum, dubnium, chromium, molybdenum, tungsten, seaborgium, magnesium, technetium, rhenium, bohrium, iron, ruthenium, osmium, and hassium. In an embodiment, the inorganic and/or organometallic complex includes one or more of Ta, Nb, V, Mo, W, Cr, Re, Rh and Ru. In an embodiment, the inorganic and/or organometallic complex includes one or more of Mo, W, Cr, and Re.

In an embodiment, the present invention relates to a single site catalyst and/or a single site precatalyst. In another embodiment, the present invention relates to a catalyst and/or a precatalyst comprising inorganic and/or organometallic complexes of group V or VI or VII elements grafted on inorganic oxide and/or on silicon modified inorganic oxide and/or on bismuth modified inorganic oxide; preferably Mo, W, Cr and/or Rhenium grafted on inorganic oxide and/or on silicon modified inorganic oxide and/or on bismuth modified inorganic oxide.

In an embodiment, the present invention relates to supported Monopodal and/or Bipodal (single site) catalysts and/or precatalysts.

In an embodiment, the present invention relates to supported Monopodal and/or Bipodal (single site) catalysts and/or precatalysts which are monometallic and/or bimetallic; wherein monometallic means organometallic complexes having only one metal center surrounded by ligands, and bimetallic means organometallic complexes in which two metal centers are directly connected or bridged via an element, such as oxygen.

In an embodiment, the present invention relates to supported Monopodal (single site) catalysts and/or precatalysts, i.e. catalysts or precatalysts in which one bond of the metal is anchored to the support through one oxygen atom.

In an embodiment, the present invention relates to supported Bipodal (single site) catalysts and/or precatalysts, i.e. catalysts or precatalysts in which two bonds of the metal are anchored to the support, each bond being anchored through one oxygen atom.

### Bipodal Catalysts

In a preferred embodiment, the present invention relates to supported Bipodal (single site) catalysts and/or precatalysts, selected from Bipodal tetrahedral catalysts and/or precatalysts, from Bipodal pentahedral catalysts and/or precatalysts, and/or from Bipodal hexahedral catalysts and/or precatalysts.

Examples of said supported Bipodal (single site) catalysts and/or precatalysts are defined and illustrated herein below. In these examples, “M₁” denote silicon and/or bismuth which are part of the oxide support, and “Me” denotes a metal of group VI elements (W, Cr, Mo, preferably molybdenum):

BIPO1—Bipodal tetrahedral monometallic or bimetallic (single site) catalysts and/or precatalysts: The metallic site is four coordinated in which two bonds are anchored to the support through an oxygen atom and

BIPO1.1. the remaining ligands are preferably selected from oxo and/or imido groups as illustratively represented by

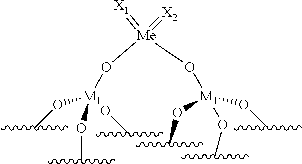

wherein X₁ and X₂ are the same or different and are selected from O, NH, and/or NR, wherein R is selected from alkyl (e.g., trimethylsilyl and/or tertiobutyl) and/or aryl (e.g., 2,6-isopropylphenyl), tris-alkylsilyl, tris-arylsilyl, tris-alkylstanyl, and/or tris-arylstanyl; and/or

BIPO1.2. the remaining ligands are preferably selected from carbyne or substituted carbyne together with alkyl, aryl, neosilyl, hydrogen, alkoxy, aryloxy, and/or amide as illustratively represented by

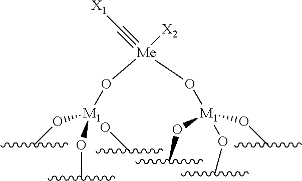

wherein X₁ and X₂ are the same or different and are selected from alkyl, aryl trimethylsilyl, H, alkoxy and/or aryloxy; preferably wherein X₁ is selected from alkyl, aryl, trimethylsilyl, and/or H, and X₂ is selected from alkyl (e.g. methyl, neosilyl, neopentyl, neophyl, and/or benzyl), aryl, alkoxy, aryloxy, thio-aryloxy, and/or amide; and/or

BIPO1.3. the remaining ligands are preferably selected from allyl, substituted allyl, aryl, alkoxy, aryloxy, and/or amide as illustratively represented by

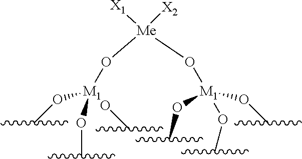

wherein X1 and X2 are the same or different and are selected from allyl, substituted allyl, aryl, alkoxy, aryloxy, thio-aryloxy, and/or amide; and/or

BIPO1.4. the remaining ligands are preferably selected from metal triple bond together with alkyl (e.g. neosilyl, neopentyl. neophyl, and/or benzyl), aryl, alkoxy, aryloxy, and/or amides as illustratively represented by

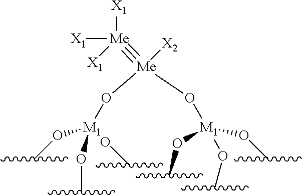

wherein X₁ and X₂ are the same or different and are selected from alkyl (e.g. neosilyl, neopentyl, neophyl, and/or benzyl), aryl, alkoxy, aryloxy and/or amide.

BIPO2—Bipodal pentahedral (single site) catalysts and/or recatalsts: The metallic site is penta-coordinated monomeric or dimeric in which two bonds are anchored to the support through an oxygen atom and the remaining ligands are preferably selected from oxo and/or imido and/or carbene groups together with alkyl (e.g. neosilyl, neopentyl, neophyl, benzyl), aryl, alkoxy, aryloxy, thio-aryloxy, amides, pyrolidyl, substituted pyrolidyl, and/or sulfides as illustratively represented by

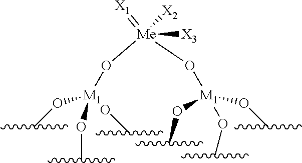

wherein X₁ is selected from O, and/or NH, and/or NR and/or CHR, wherein R is selected from alkyl (e.g., Trimethylsilyl and/or tertiobutyl) and/or aryl (e.g., 2,6-isopropylphenyl), and wherein X₂ and X₃ are the same or different and are selected from alkyl (e.g. methyl, neopentyl, neosilyl, benzyl and/or neophyl), aryl, alkoxy, aryloxy, siloxy, thio-aryloxy, amide, pyrolidyl, and/or substituted pyrolidyl.

BIPO2DIM—Bipodal pentahedral dimeric (single site) catalysts and/or precatalysts: The metallic site is penta-coordinated in which two bonds are anchored to the support through an oxygen atom and the remaining ligands are preferably selected from oxo groups together with alkyls and/or aryl and/or aryloxy and/or thioaryloxy and/or siloxy and/or amide and/or pyrolidyl and/or substituted pyrolidyl and metal oxo ligand as illustratively represented by

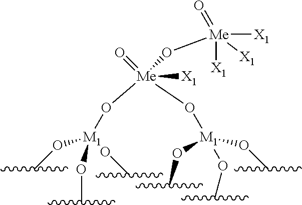

wherein X₁ is selected from alkyl (e.g. methyl, neopentyl, neosilyl, benzyl, and/or neophyl), aryl, alkoxy, aryloxy, siloxy, thio-aryloxy, amide, pyrolidyl, and/or substituted pyrolidyl.

BIPO3—Bipodal hexahedral (single site) catalysts and/or precatalysts: The metallic site is hexa-coordinated in which two bonds are anchored to the support through an oxygen atom and the remaining ligands are preferably selected from one or more of alkyls, aryls, alkoxys, aryloxys, thio-aryloxys, amides, and sulfides, as illustratively represented by

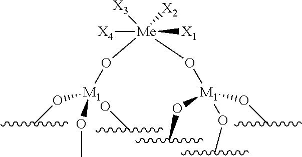

wherein X₁, X₂, X₃, and X₄ are the same or different and are selected from alkyl (e.g. methyl, neopentyl, neosilyl, benzyl, and/or neophyl), aryl, alkoxy, aryloxy, thio-aryloxy, siloxy, and/or amide.

### Monopodal Catalysts

In an alternative and/or additional preferred embodiment, the present invention also relates to supported Monopodal (single site) catalysts and/or precatalysts, selected from Monopodal (e.g. monometallic or bimetallic) tetrahedral catalysts and/or precatalysts, from Monopodal (e.g. monometallic or bimetallic) pentahedral catalysts and/or precatalysts, and/or from Monopodal hexahedral catalysts and/or precatalysts.

Examples of said supported Monopodal (single site) catalysts and/or precatalysts are defined and illustrated herein below:

MONO1—Monopodal tetrahedral (single site) catalysts and/or precatalysts: The metallic site is four coordinated in which one bond is anchored to the support through an oxygen atom and

MONO1.1. the remaining ligands are preferably selected from oxo and/or imido and/or carbene derivatives together with alkyl, alkoxy, aryloxy, thioaryloxy, amide and/or pyrolidyl as illustratively represented by

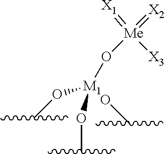

wherein X₁ and X₂ are the same or different and are selected from O, NH, and/or NR wherein R is selected from alkyl and/or aryl, and X₃ is selected from alkyl (e.g. neosilyl, neopentyl. neophyl, and/or benzyl), aryl (e.g. Mesityl), alkoxy, aryloxy, thio-aryloxy, siloxy, and/or amide; and/or

MONO1.2. the remaining ligands are preferably selected as illustratively represented by

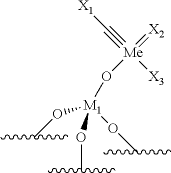

wherein X₁ is selected from alkyl, aryl, and/or H, and X₂ and X₃ are the same or different and are selected from alkyl (e.g. methyl, neosilyl, neopentyl, neophyl, and/or benzyl), aryl, alkoxy, aryloxy, thio-aryloxy, siloxy, and/or amide; and/or

MONO1.3. the remaining ligands are preferably selected as illustratively represented by

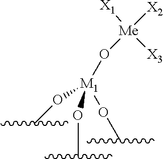

wherein X₁, X₂ and X₃ are the same or different and are selected from allyl and/or substituted allyl and/or aryl and/or alkoxy and/or aryloxy and/or thio-aryloxy and/or siloxy and/or amide; and/or

MONO1.4-DIM—the remaining ligands are preferably selected from metal triple bond together with alkyl (e.g. neosilyl, neopentyl, neophyl, and/or benzyl), aryl (e.g. Mesityl), alkoxy, aryloxy, and/or amides as illustratively represented by

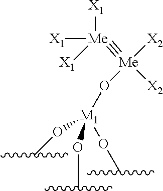

wherein X₁ and X₂ are the same or different and are selected from allyl and/or substituted allyl and/or aryl and/or alkoxy and/or aryloxy and/or thio-aryloxy and/or siloxy and/or amide.

MONO2—Monovodal pentahedral (single site) catalysts and/or precatalysts: The metallic site is penta-coordinated in which one bonds is anchored to the support through an oxygen atom and the remaining ligands are preferably selected from oxo and/or imido and/or carbene groups together with alkyls (eg. neosilyl, neopentyl neophyl, benzyl) and/or alkoxy and/or aryloxy and/or thio-aryloxy and/or amides and/or pyrolidyl and/or substituted pyrolidyl and/or sulfides as illustratively represented by

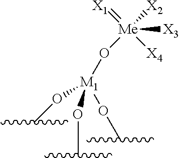

wherein X₁ is selected from O, NH, NR, and/or CHR, wherein R is selected from alkyl (e.g. Trimethylsilyl and/or tertiobutyl) and/or aryl, and wherein X₂, X₃ and X₄ are the same or different and are selected from alkyl (e.g. methyl, neopentyl, neosilyl, benzyl, and/or neophyl) and/or aryl (e.g. Mesityl) and/or alkoxy and/or aryloxy and/or thio-aryloxy and/or and/or siloxy and/or amide and/or pyrolidyl and/or substituted pyrolidyl.

MONO2DIM—Monopodal pentahedral dimeric (single site) catalysts and/or precatalysts: The metallic site is penta-coordinated in which one bond is anchored to the support and the remaining ligands are preferably selected from oxo groups together with alkyls and/or aryl and/or aryloxy and/or thioaryloxy and/or siloxy and/or amide and/or pyrolidyl and/or substituted pyrolidyl and metal oxo ligand as illustratively represented b

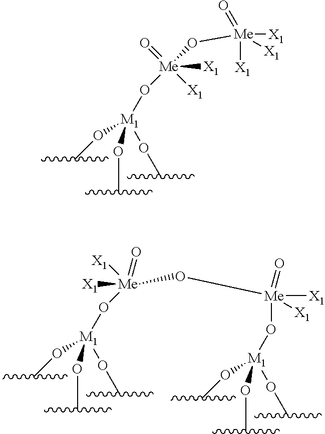

wherein X₁ is selected from alkyl (e.g. methyl, neopentyl, neosilyl, benzyl, and/or neophyl) and/or aryl and/or alkoxy and/or aryloxy and/or siloxy and/or thio-aryloxy and/or amide and/or pyrolidyl and/or substituted pyrolidyl.

MONO3—Monopodal hexahedral (single site) catalysts and/or Precatalysts: The metallic site is hexa-coordinated in which two bonds are anchored to the support through an atom of oxygen and the remaining ligands can be alkyl (e.g. neophyl, neopentyl, benzyl, neosilyl) and/or aryl and/or alkoxy and/or amides as illustratively represented by

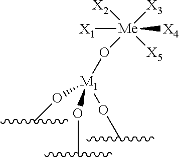

wherein X₁, X₂, X₃, X₄ and X₅ are the same or different and are selected from R wherein R is selected from alkyl (e.g. methyl, neopentyl, neosilyl, benzyl, and/or neophyl) and/or aryl and/or alkoxy and/or aryloxy and/or siloxy and/or thio-aryloxy, and/or amide.

In an embodiment of the present invention, the claimed catalysts and/or precatalysts, in particular single site catalysts and/or a single site precatalysts, comprising inorganic and/or organic metallic complexes are characterized in that the metal is selected from molybdenum, tungsten, chromium and/or rhenium (Mo, W, Cr and/or Re).

In an embodiment of the present invention, the claimed supported catalysts and/or precatalysts, in particular single site catalysts (and/or precatalysts) comprising inorganic and/or organometallic complexes are characterized in that the metals of the inorganic and/or organometallic complexes have one and/or two bonds which are anchored to the support, each bond being anchored via an oxygen atom.

In all the above illustrative figures, M1 of the support can advantageously represent silicon, bismuth, aluminum, titanium, zirconium, cerium, magnesium and/or mixtures thereof.

In an embodiment of the present invention, the claimed catalysts and/or precatalysts, in particular single site catalysts and/or single site precatalysts, comprising inorganic and/or organic metallic complexes are characterized in that the inorganic and/or organic metallic complexes are selected from
metal-bis-oxo or metal-bis-oxo and alkyl and/or aryl and/or alkoxy and/or aryloxy and/or thio-aryloxy and/or siloxide and/or amides,
metal-bis-imido or metal-bis-imido and alkyl and/or aryl and/or alkoxy and/or aryloxy and/or thio-aryloxy and/or siloxide and/or amides,
metal-carbene and alkyl and/or aryl and/or alkoxy and/or siloxide and/or amides and/or pyrolidyl and/or thioaryloxide and/or aryloxy and/or thio-aryloxy,
metal-imido and alkyl and/or aryl and/or alkoxy and/or siloxide and/or aryloxy and/or amides and/or pyrolidyl and/or thioaryloxy,
metal-oxo-and alkyl and/or halide (e.g. chloride, bromide, fluoride) and/or aryl and/or pyrolidyl and/or alkoxide and/or siloxide and/or aryloxide and/or thioaryloxide and/or amides,
metal tetra-alkoxy and/or -amides and/or -allyl and/or -allyl-substituted,
metal hexa-amides and/or -alkoxide and/or -aryloxide,
metal carbyne and alkyl and/or alkoxide and/or aryloxide and/or siloxide and/or amides and/or thioaryloxide,
metal≡metal triple bond and alkyl and/or aryloxide and/or alkoxide and/or amides and/or thioaryloxide and/or siloxide.

In an embodiment of the present invention, the claimed single site catalyst and/or a single site catalyst precatalyst comprising inorganic and/or organic metallic complexes are characterized in that the content of the active metal (preferably selected from Mo, W, Cr and/or Re) is lower than 25 wt %, for example lower than 20 wt %, preferably lower than 15 wt %, for example lower than 12 wt %. This concentration of metal can for example be measured by elemental analysis.

In an embodiment, catalysts and/or precatalysts bearing bis-oxo and/or bis-imido substitutes supported on SiO₂₍₂₀₀₎ surface may be characterized by one or more of the following:

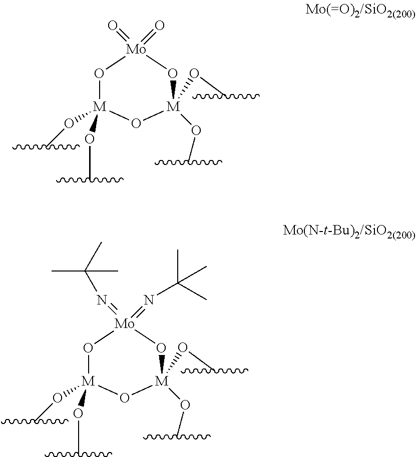

In an embodiment, catalysts and/or precatalysts bearing bis-imido substituents supported on SiO₂Bi₂O₃₍₂₀₀₎ surface may be characterized by one or more of the following:

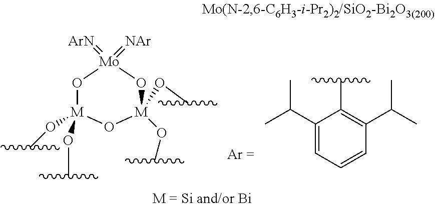

where Mo content is about 2.31% wt and Bi content is about 10.4% wt.

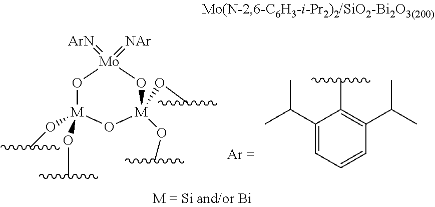

where Mo content is about 1.49% wt and Bi content is about 10.0% wt.

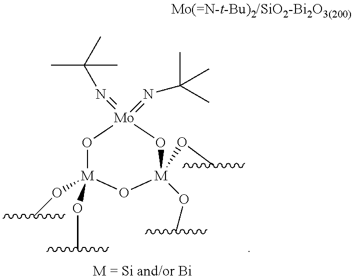

In an embodiment, catalysts and/or precatalysts bearing carbene/carbyne/oxo-alkyl substituents supported on SiO₂.Bi₂O₃₍₂₀₀₎ surface may be characterized by one or more of the following:

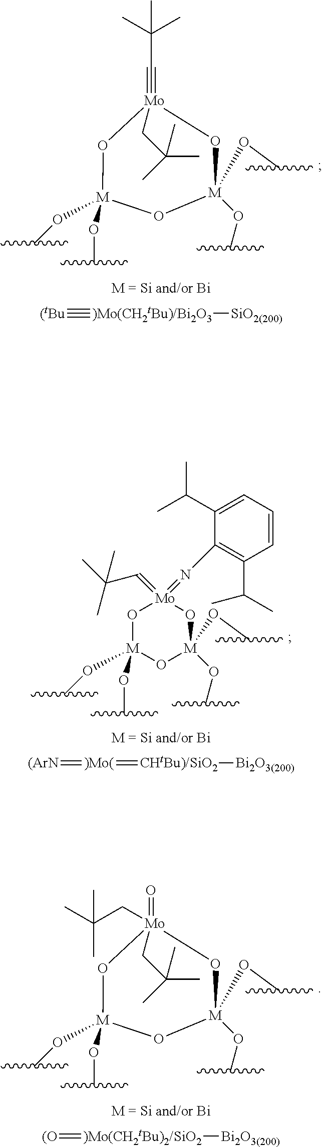

A preferred catalyst and/or precatalyst according to the present invention can advantageously be represented by the following formula illustratively represented in the table below wherein “Si” and “M₁” denote silicon and/or bismuth which are part of the oxide support, and “Me” denotes metal (e.g. W, Cr, Mo, preferably molybdenum).

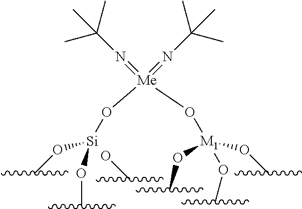

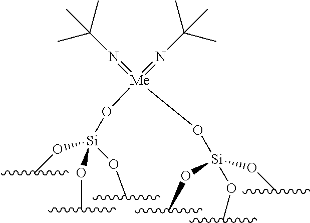

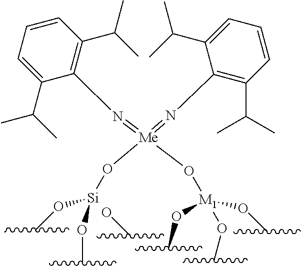

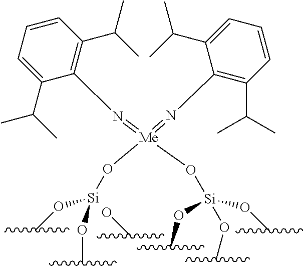

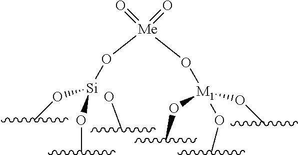

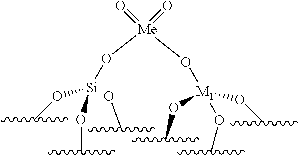

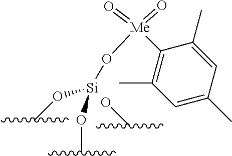

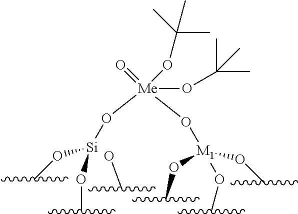

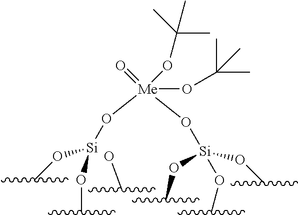

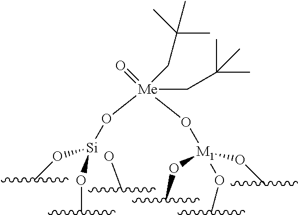

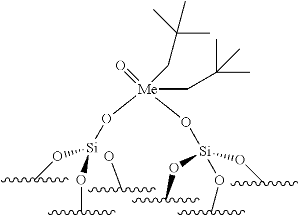

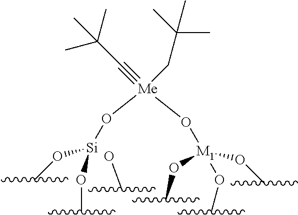

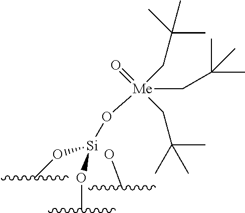

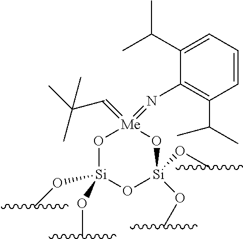

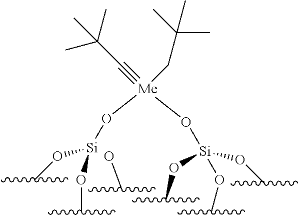

In addition, the Applicants believe that some of these solid metal complexes were never disclosed in the prior art. The Applicants have also unexpectedly found that these solid metal complexes could be used as catalysts, in particular as catalyst useful for the conversion of olefins to aldehydes, nitriles and/or acrylics, more particularly for the conversion of propylene
in the presence of oxygen to ethanal and/or propenal, and
in the additional presence of ammonia to ethanal and/or propenal and/or acrylonitrile and/or acetonitrile.

### Support

The synthesis of the supported metal complexes may be conducted on inorganic oxide supports. The support may include any inorganic oxide supports. In many embodiments, the support includes one or more of inorganic oxide, silicon-modified inorganic oxide, and bismuth-modified inorganic oxide. In preferred embodiments, the support may include one or more of silica, bismuth oxide, bismuth-modified silica, and silicon-modified bismuth oxide. In other embodiments, the support may additionally and/or alternatively include one or more of silica, fibrous silica, bismuth oxide, bismuth-modified silica, silicon-modified bismuth oxide, alumina, titania, magnesia, ceria, alumino-silicates, clays, zeolites (e.g. any kind of zeolite, including hierarchical zeolites), ceria, fibrous silica such as KCC 1 mesoporous zeolites or mesoporous any kind of these oxides.

The support may be characterized by a BET surface area. For example, in many embodiments, a specific surface area (BET) of the support may range from about 50 m²/g to about 1200 m²/g. In preferred embodiments, a specific surface area (BET of the support may range from about 100 m²/g to 500 m²/g. In more preferred embodiments, a specific surface area (BET of the support may range from about 125 m²/g to 350 m²/g. The specific surface area (B.E.T.) is measured according to the standard ISO 9277 (1995). The support may be one or more of predominately macroporous, predominately microporous, and predominately mesoporous.

The support may be provided (e.g., physically) in any suitable form. For example, the support may be provided as a powder, extrude, and/or a variety of other catalytic shapes. The final compound may be sufficiently stable to allow molding or palletization of the final catalyst; during this stage a binder may optionally be added.

In an embodiment, the support is silica. For example, the silica may include silicon oxide that is substantially free from any other oxide and/or contains less than about 2 wt % of one or more other oxides, which may be present in the form of impurities.

In an embodiment, the support is bismuth oxide. For example, the bismuth oxide may include bismuth oxide that is substantially free from any other oxide and/or contains less than about 2 wt % of one or more other oxides, which may be present in the form of impurities.

In an embodiment, the support is bismuth-modified silica. For example, the bismuth-modified silica may include bismuth oxide doped silica including at least about 2.5 wt % of bismuth, at least about 5 wt % bismuth, and/or greater than about 8 wt % bismuth. In addition, or in the alternative, the bismuth oxide doped silica includes less than about 50 wt % bismuth, less than about 25 wt % bismuth, and/or less than about 15 wt % bismuth.

In an embodiment, the support is silicon-modified bismuth oxide. For example, the silicon-modified bismuth oxide may include silicon oxide doped bismuth oxide including at least about 0.5 wt % of silicon, at least about 2.5 wt % of silicon, and/or greater than about 8 wt % silicon. In addition, or in the alternative, the silicon oxide doped bismuth oxide may include less than about 50 wt % silicon, less than about 25 wt % silicon, and/or less than about 15 wt % silicon.

In an embodiment, the support (e.g., before use in the preparation process of the present invention) may be analyzed to determine a hydroxyl content. The hydroxyl content may range from about 0.3 to about 8.0 OH/nm². For example, in an embodiment, a hydroxyl content may range from about 0.5 to about 4.0 OH/nm², as determined by, for example, titration by CH₃MgBr and ¹H solid state NMR. The support may be subjected to a so-called “activation” treatment which can advantageously include a thermal (or dehydration) treatment. The said activation treatment makes it possible to remove at least some of the water contained in the support catalyst/precatalyst, and also partially the hydroxyl groups, thus allowing some residual hydroxyl groups and a specific porous structure to remain. The choice of the support catalyst/precatalyst may impact the conditions of the activation treatment, e.g. the temperature and the pressure, in order to fulfill the above final support characteristics. This may be defined on a case-by-case basis depending on the selection of the catalyst/precatalyst and its reaction to the activation treatment. For example, the activation treatment may be carried out under a current of air or another gas, particularly an inert gas, e.g. nitrogen, as well as under reduced pressure (from low vacuum to ultra-high vacuum, preferably under high vacuum), at a temperature chosen from about 50 to about 1000° C., or preferably from about 100 to about 900° C.

An example of a silica support that may be used in the present invention is described in Prof. Dr. Jean Marie Basset's publication (Angewandte Chemie International Edition, Volume 49, Issue 50, Dec. 10, 2010, Pages 9652-9656, “High-Surface-Area Silica Nanospheres (KCC-1) with a Fibrous Morphology”).

According to an embodiment of the present invention, the hydroxyl content of the support of the monopodal single-site catalysts and/or precatalysts may be lower than the hydroxyl content of the bipodal single-site catalysts and/or precatalysts.

For example, the synthesis of the monopodal (single site) catalysts and/or precatalysts may be favored when the hydroxyl content of the support is lower than about 1.5 OH/nm², (as determined by titration and ¹H solid state NMR). For example, the synthesis of the monopodal (single site) catalysts and/or precatalysts may be favored when the support is subjected to an activation treatment as defined above at a temperature higher than about 350° C., e.g. chosen from about 400 to about 1000° C.

For example, the synthesis of the bipodal (single site) catalysts and/or precatalysts may be favored when the hydroxyl content of the support is higher than about 1.5 OH/nm², e.g. comprised between about 1.8 and about 4 OH/nm² (as determined by titration and ¹H solid state NMR). For example, the synthesis of the bipodal (single site) catalysts and/or precatalysts may be favored when the support is subjected to an activation treatment as defined above at a temperature lower than about 350° C., e.g. chosen from about 50 to about 300° C., or even lower than about 250° C.

Examples of synthesis can advantageously be found in Y Bouhoute et al. ACS Catal., 2016, 6 (1), pp 1-18 (“Accessing Realistic Models for the WO₃—SiO₂ Industrial Catalyst through the Design of Organometallic Precursors”) and/or in N Merle et al. J Am Chem Soc 2017, 139 (6), 2144-2147 (“Well-Defined Molybdenum Oxo Alkyl Complex Supported on Silica by Surface Organometallic Chemistry: A Highly Active Olefin Metathesis Precatalyst”).

### Process

### Oxidation and Ammoxidation of Olefins to Aldehydes by Molecular Oxygen Using a Single-Site Olefin Metathesis Catalyst

In an embodiment, the present invention relates to a process for manufacturing aldehydes/nitriles from an olefin which comprises the reaction of an olefin in the presence of oxygen, optionally ammonia, and a catalyst and/or a precatalyst, in particular a single site catalyst or precatalyst.

In an embodiment, the present invention relates to a process for manufacturing aldehydes and/or nitriles from an olefin which comprises the reaction of an olefin in the presence of oxygen, optionally ammonia, and a catalyst and/or a precatalyst comprising inorganic and/or organometallic complexes of group V or VI or VII elements grafted on inorganic oxide and/or on silicon modified inorganic oxide and/or on bismuth modified inorganic oxide.

In a preferred embodiment, the present invention relates to a process for manufacturing aldehydes/nitriles from an olefin which comprises the reaction of an olefin in the presence of oxygen, optionally ammonia, and a supported metal compound catalyst characterized in that the said supported metal compound catalyst comprises a supported metal complex as defined herein below.

In an embodiment of the present invention, the process for manufacturing aldehydes/nitriles from an olefin is conducted in gas-phase, for example in a gas phase reactor which can advantageously be selected amongst fixed-bed flow reactor or fluidized bed reactor.

In an embodiment of the present invention, the process for manufacturing aldehydes/nitriles from an olefin is conducted at a temperature superior to 25° C., preferably superior to 200° C., for example superior to 350° C. In an embodiment of the present invention, the process for manufacturing aldehydes/nitriles from an olefin is conducted at a temperature lower than 600° C., preferably lower than 500° C., for example lower than 480° C.

In an embodiment of the present invention, the process for manufacturing aldehydes/nitriles from an olefin is conducted at a total absolute pressure, chosen in a range of from 0.01 to 50 MPa, preferably from 0.1 to 15 MPa, in particular from 0.1 to 10 MPa.

The exact content of the olefin reacting gas mixture will advantageously be selected depending on the oxidation and/or ammoxidation objectives.

In an embodiment according to the present invention, the olefin will be selected amongst olefinic hydrocarbons having 2 to 4 carbon atoms, for example linear alkenes (especially propylene or butenes) or branched alkenes e.g. having an “iso” structure (especially isobutene), or a mixture of two or more of the said olefins, preferably propylene. In an embodiment, the olefin is selected from propylene, isobutene, or a mixture thereof.

The exact content of the olefin in the olefin reacting gas mixture will advantageously be selected depending on the oxidation and/or ammoxidation objectives.

In an embodiment according to the present invention, the reacting olefin will represent at least 0.5 volume percent of the olefin reacting gas mixture, preferably at least 1.5 volume percent of the olefin reacting gas mixture, for example at least 5 volume percent of the olefin reacting gas mixture. In an embodiment according to the present invention, the reacting olefin will represent less than 50 volume percent, for example less than 25 volume percent of the olefin reacting gas mixture.

In an embodiment according to the present invention, the reacting oxidant will be selected amongst oxygen and/or air and will advantageously represent at least 0.5 volume percent of the olefin reacting gas mixture, preferably at least 1.5 volume percent of the olefin reacting gas mixture, for example at least 5 volume percent of the olefin reacting gas mixture. In an embodiment according to the present invention, the reacting oxidant will represent less than 25 volume percent, for example less than 15 volume percent of the olefin reacting gas mixture.

In an embodiment according to the present invention, for the ammoxidation reaction process, the olefin reacting gas mixture will comprise a nitrogen reacting compound, for example ammonia. Said nitrogen reacting compound will advantageously represent at least 0.5 volume percent of the olefin reacting gas mixture, preferably at least 1.5 volume percent of the olefin reacting gas mixture, for example at least 5 volume percent of the olefin reacting gas mixture. In an embodiment according to the present invention, the nitrogen reacting compound will represent less than 25 volume percent, for example less than 15 volume percent of the olefin reacting gas mixture.

In an embodiment according to the present invention, the remaining constituents of the olefin reacting gas mixture will preferably be selected amongst inert gases, e.g. nitrogen, helium, argon or mixtures thereof.

In an embodiment according to the present invention, the catalytic bed may be diluted, e.g. by mixing intimately the catalyst with inactive ceramic bodies diluents without affecting the fluid flow through the catalyst bed; illustrative examples of said diluents are SiO₂ (e.g. silica sand, fused silica, . . . ), quartz (e.g. quartz chips), SiC, alpha-alumina, glass beads, preferably SiC or quartz.

In an embodiment, the aldehydes are selected from ethanal, propanal, propenal, and mixtures thereof. In an embodiment, ammonia is present and the nitriles are selected from acrylonitrile, acetonitrile, and mixtures thereof. In an embodiment, the catalyst and/or precatalyst has a content of active metal (preferably selected from Mo, W, Cr and/or Re) which is lower than 20 wt %, preferably lower than 15 wt %, for example lower than 12 wt %. In an embodiment, the support of the catalyst and/or precatalyst is selected from silica, bismuth oxide, bismuth modified silica, and/or silicon modified bismuth oxide. In an embodiment, the catalyst and/or precatalyst may be used to produce ethanal, propanal, propenal, acetonitrile, acrylonitrile, or a mixture of two or more thereof.

In an embodiment, a method of making aldehydes and/or nitriles may comprise contacting an olefin with a supported single-site catalyst in a presence of one or more of oxygen and ammonia to produce one or more of aldehydes and nitriles.

In an embodiment, a method of making one or more of aldehydes and nitriles may comprise contacting an olefin and one or more of oxygen and ammonia in a presence of a catalyst to produce one or more of aldehydes and nitriles. In an embodiment, the catalyst is a single-site catalyst including an inorganic and/or organometallic complex grafted on a support.

The method may be used to make aldehydes and/or nitriles on any scale, such as scaled-up industrial processes. For example, the method may be used as a process for manufacturing aldehydes and/or nitriles, among other things.

The method includes contacting an olefin with a supported single-site catalyst in a presence of one or more of oxygen and ammonia to produce one or more of aldehydes and nitriles. In this step, a reaction mixture in any phase (e.g., gas, vapor, liquid, solid, gel, etc.) is brought into physical contact with, or immediate or close proximity to, the supported single-site catalyst. The contacting may include one or more of feeding, flowing, and passing the reaction mixture sufficient to bring the reaction mixture into contact with the supported single-site catalyst. The contacting may proceed in a batch or continuous process. The contacting may proceed in any suitable reactor, such as a continuous flow reactor. In many embodiments, the contacting may proceed in a gas-phase reactor, including, but not limited to, one or more of a fixed-bed flow reactor and fluidized bed reactor, among other types of reactors known in the art.

The contacting may proceed at and/or under reaction conditions suitable to produce one or more of aldehydes and nitriles. For example, the reaction conditions may be suitable for one or more of oxidation and ammoxidation. The reactions conditions may include one or more of temperature and pressure, among others.

The contacting may proceed at or to a temperature ranging from about 25° C. to about 600° C. In an embodiment, the contacting may proceed at or to a temperature that is greater than about 25° C. In an embodiment, the contacting may proceed at or to a temperature that is greater than about 200° C. In an embodiment, the contacting may proceed at or to a temperature that is greater than about 350° C. In an embodiment, the contacting may proceed at or to a temperature that is less than about 600° C. In an embodiment, the contacting may proceed at or to a temperature that is less than about 500° C. In an embodiment, the contacting may proceed at or to a temperature that is less than about 480° C. In many embodiments, the contacting may proceed at or to a temperature of about 400° C. In preferred embodiments, the contacting may proceed at or to a temperature of about 450° C. In other embodiments, the contacting may proceed at or to a temperature that is less than about 25° C. and/or greater than about 600° C.

The contacting may proceed at a pressure ranging from about 0.01 MPa to about 50 MPa. In an embodiment, the contacting may proceed at a pressure ranging from about 0.1 MPa to about 15 MPa. In an embodiment, the contacting may proceed at a pressure ranging from about 0.1 MPa to about 10 MPa. In other embodiments, the contacting may proceed at a pressure that is less than about 0.01 MPa and/or greater than about 50 MPa.

The reaction mixture may include the olefin, one or more oxidants, and/or one or more inert species. One or more of the olefin and oxidants may be present in the reaction mixture in any phase. In many embodiments, one or more of the olefin and oxidants are present in the reaction mixture in a gas and/or vapor phase.

The olefin may include one or more of terminal olefins and internal olefins.

The olefin may include an olefinic hydrocarbon having about 2 to about 4 carbons. The olefins may include one or more of linear alkenes (e.g., propylene and butenes) and branched alkenes (e.g., alkenes with an “iso” structure, such as isobutenes). The olefins may include a single olefin species and/or a mixture of olefin species. In many embodiments, the olefin includes one or more of propylene, isobutene, 2-butenes, 1-butene, and 2-pentenes.

The oxidant may include any element and/or compound including one or more of an oxygen and a nitrogen. For example, the oxidant may include one or more of an oxygen-containing compound and a nitrogen-containing compound. In an embodiment, the oxidant may include air. In an embodiment, the oxidant may include molecular oxygen (e.g., O₂). In an embodiment, the oxidant may include ammonia. In an embodiment, the oxidant may include one or more of air and ammonia. In an embodiment, the oxidant may include one or more of oxygen and ammonia. In an embodiment, the oxidant may include one or more of molecular oxygen and ammonia.

The aldehyde may include any chemical species including a —CHO group. For example, the aldehyde may include one or more of ethanal, propanal, and propenal. The nitrile may include any chemical species including a —CN group. For example, the nitrile may include one or more of acrylonitrile and acetonitrile.

### Metathetic Oxidation of Olefins to Aldehydes by Molecular Oxygen Using a Bis-Oxo Molybdenum Single-Site Olefin Metathesis Catalyst

In an embodiment, the present invention relates to a process relating to a direct catalytic oxidation of internal olefins and/or α-olefins to aldehydes via single-step catalysis using molecular oxygen by metathesis.

In an embodiment, the present invention relates to a process comprising contacting an olefin and oxygen in a presence of a catalyst to produce aldehydes.

In an embodiment, the contacting gives rise to a reaction in which a double bond of an olefin reacts with a double bond of oxygen (e.g., molecular oxygen) to form acetaldehyde during metathetic oxidation. In an embodiment, the contacting may proceed at or to any conditions suitable for metathetic oxidation. For example, in an embodiment, the contacting may proceed at or to a temperature ranging from about 300° C. to about 500° C. In a preferred embodiment, the contacting may proceed at or to a temperature ranging from about 350° C. to about 450° C. In a preferred embodiment, the contacting may proceed at or to a temperature ranging from about 400° C. to about 450° C. In a more preferred embodiment, the contacting may proceed at about 450° C.

In an embodiment, a ratio of oxygen to olefin may range from 1:100 to 100:1. In an embodiment, a ratio of oxygen to olefin may be about 2.5:7.5, about 5:7.5; and/or about 10:7.5, among others.

In an embodiment, the oxygen is molecular oxygen (e.g., O₂). In an embodiment, the olefin may include any internal olefin and/or α-olefin. In a preferred embodiment, the olefin includes propylene. In a preferred embodiment, the olefin includes 2-butenes. In a preferred embodiment, the olefin includes cis-2-butene. In a preferred embodiment, the olefin includes cis-2-pentene.

In an embodiment, the catalyst includes a metathetic oxidation catalyst. In an embodiment, the catalyst includes a single-site catalyst. In an embodiment, the catalyst includes a single-site olefin metathesis catalyst. In an embodiment, the catalyst includes a Mo(bis-oxo) species. In an embodiment, the catalyst includes a supported Mo(bis-oxo) species. In an embodiment, the catalyst includes a supported bipodal Mo(bis-oxo) species. In an embodiment, the catalyst includes a bipodal Mo(bis-oxo) species supported on silica. In an embodiment, the catalyst includes a bipodal Mo(bis-oxo) species supported on silica dehydroxylated at about 200° C. (e.g., SiO₂₋₍₂₀₀₎). In an embodiment, the Mo(bis-oxo) species may be supported on any of the supports of the present disclosure. In a preferred embodiment, the catalyst includes (≡Si—O—)₂Mo(═O)₂.

In an embodiment, the process produces any aldehyde, such as one or more of ethanal (or acetaldehyde), propanal, and propenal, among others. In a preferred embodiment, the process produces ethanal. In an embodiment, the process may additional produce one or more of CO, CO₂, acrolein, formaldehyde, acetone, ethylene, and propylene oxide.

In an embodiment, a conversion of the olefin may range from about greater than 0% to about 20%. For example, in an embodiment, a conversion of the olefin may be about 10%. In an embodiment, an acetaldehyde selectivity may range from about 20% to about 80%. For example, in an embodiment, an acetaldehyde selectivity may be about 70%.

### Process for the Preparation of the Supported Metal Complex

The first step of this preparation method is based on the preparation of the inorganic oxide supports and/or of the bismuth modified inorganic oxide supports.

For example, the introduction of bismuth oxide can be obtained either by classical impregnation or by decomposition of Bismuth precursors (eg. Bi(NO₃)₃ or BiCl₃ or BiBr₃ or Bi(OAc)₃ or Bi(2-ethylhexanoate)₃ or Bi(OPh)₃ or Bi(OtBu)₃) with an inorganic support followed by calcination of resulting materials for example at high temperature, e.g. between 250° C. and 600° C.

The second step consists on the grafting of the inorganic and/or organometallic complexes of group VI (Mo, W and Cr) or Rhenium on the inorganic oxide support and/or on the bismuth modified inorganic oxide supports and/or on the silicon modified inorganic oxide supports. This method leads to the formation of isolated metal on support with different coordination sphere containing e.g. oxo, and/or imido, and/or alkyl, and/or alkoxy, and/or aryloxy, and/or thio-aryloxy, and/or siloxide, and/or amide and/or allyl and/or pyrolidyl ligands.

The supported metal complex according to the present invention can advantageously be prepared according to the following consecutive synthesis steps:

Optional treatment of the support in order to control the hydroxyls content of the said support, said support being preferably selected amongst silica, bismuth oxide, silicon doped bismuth oxide, bismuth doped silica support, and/or a mixture of two or more of the said supports. Among the various methods employed, the so called pretreatment of the support involves a treatment under vacuum or a flowing inert gas at a temperature comprised between 100 and 900° C. and preferably 100 to 500° C. or even preferably 100 to 300° C.

### Grafting of the Metallic Complex on the Pre-Treated Support

Contact of a suitable organometallic precursor with the pre-treated support can be performed e.g. either in gas or liquid or solid phase. Solvents can advantageously be selected amongst aliphatic hydrocarbons (e.g. pentane, hexane, heptane, petroleum ether) or aromatics hydrocarbons (e.g. benzene, toluene, xylene) or polar hydrocarbons (e.g. THF, ether, dioxane, acetonitrile).

The quantity of the organometallic precursors is determined by the quantity of surface OH groups, extended from 0.1 to 20 equivalents, preferably 0.5-5, preferably 1-2.5 equivalents. The grafting reactions may be conducted at different temperature, extended from −78° C. to 250° C., preferably 0° C. to 100° C., preferably 25° C. to 50° C.

The grafting reaction may be conducted under inert atmosphere (e.g. Ar or He or N₂) or under vacuum, preferably between 10⁻¹ to 10⁻⁶ mbar.

### Example Reaction Schemes

In an embodiment, Scheme 1 is an example of a reaction scheme for preparing molybdenum bis oxo species, which may be supported/grafted on silica (e.g., silica 200) and/or bismuth:

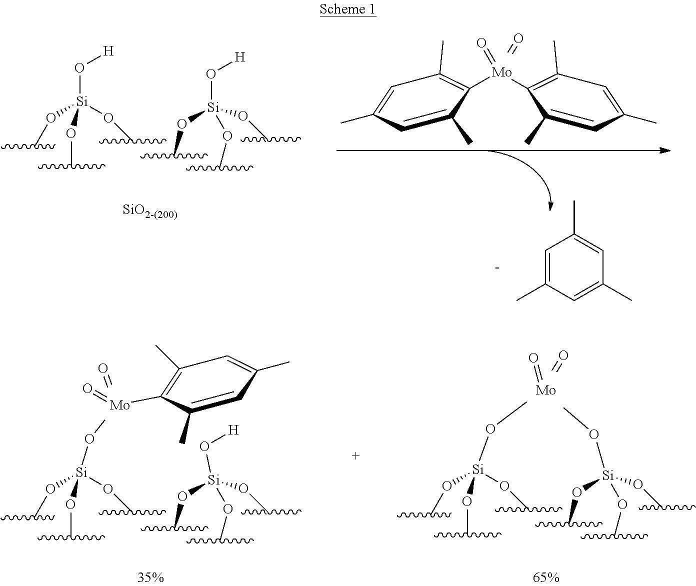

In an embodiment, Scheme 2 is an example of a reaction scheme for preparing molybdenum imido species, which may be supported/grafted on silica, bismuth, and/or silica bismuth.

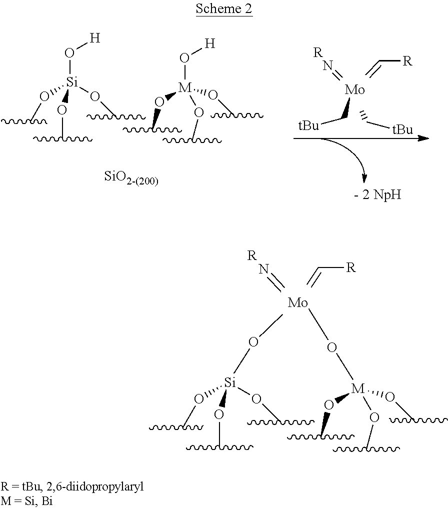

In an embodiment, Scheme 3 is an example of a reaction scheme for preparing molybdenum carbyne species supported/grafted on silica (e.g., silica 200), silica bismuth, and/or bismuth:

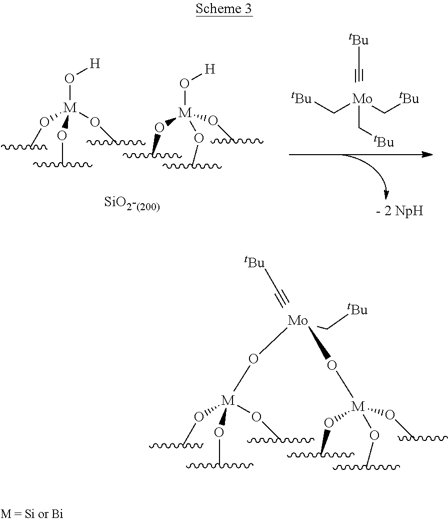

In an embodiment, Scheme 4 is an example of a reaction scheme for preparing molybdenum bis-imido species, which may be supported/grafted on silica, bismuth, and/or silica bismuth.

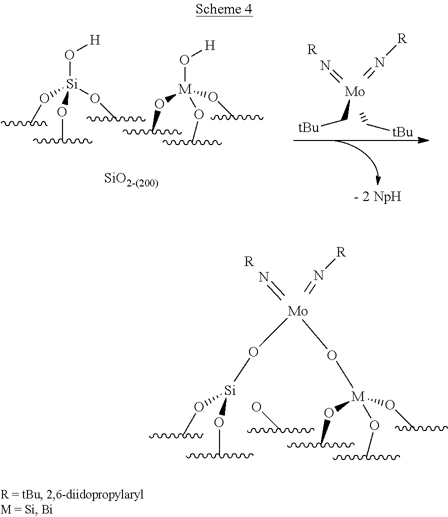

In an embodiment, Scheme 4 is an example of a reaction scheme for preparing molybdenum oxo tris-alkyl species, which may be supported/grafted onto silica (e.g., silica 200), bismuth, and/or silica bismuth.

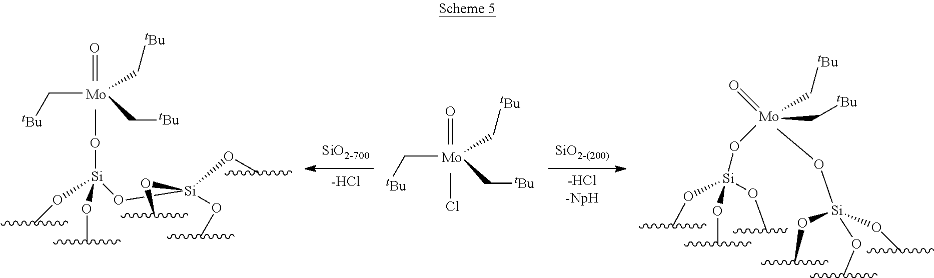

In an embodiment, Scheme 6 is an example of a reaction scheme for preparing molybdenum oxo alkoxy species, which may be supported/grafted onto silica, bismuth, and/or silica bismuth:

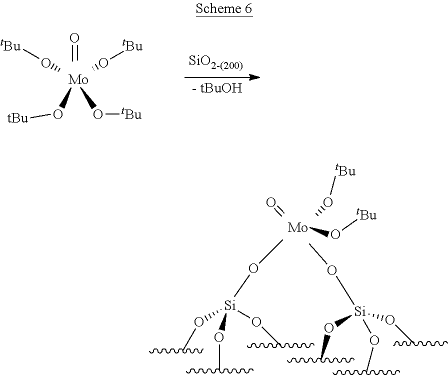

In an embodiment, Scheme 7 is an example of a reaction scheme for preparing molybdenum bis-oxo species, which may be supported/grafted onto silica, bismuth, and/or silica bismuth:

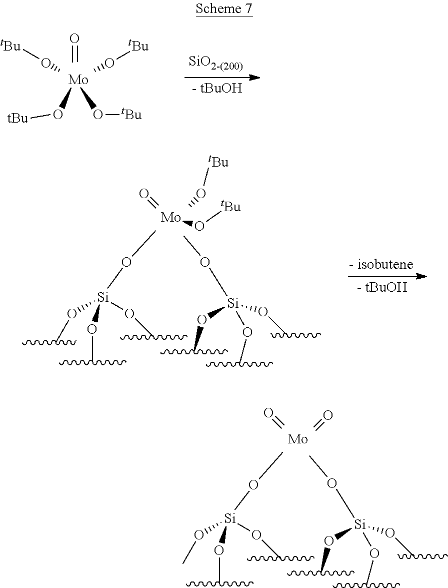

In an embodiment, Scheme 8 is an example of a reaction scheme for preparing molybdenum oxo chloride species, which may be supported/grafted onto silica, bismuth, and/or silica bismuth:

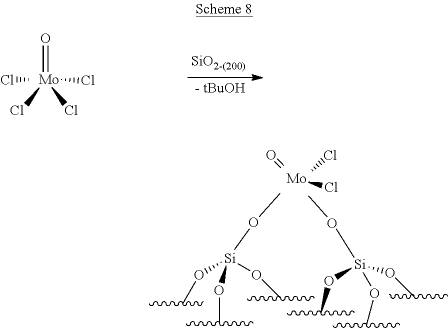

Whilst not wishing to be bound by this theory, the Applicants believe that it is a combination of the properties of the support, in particular its residual hydroxyls content, together with the metal grafting step 1, in particular the temperature at which said grafting step 1 is performed, which allows to control the formation of the respective ratios between the supported metal complex wherein the metal is anchored to the oxide support via two oxygen atoms and the supported metal complex wherein the metal is anchored to the support via one oxygen atom. This control of the ratio of the supported complexes affords fine control of the catalyst activity which is beneficial in tailoring the catalytic activity to the substrate and the desired reaction and final products. As illustrated in the examples, the process conditions and the ligands selections will also allow fine tuning the conversion process and controlling the respective ratios of final conversion products. This control is illustrated in the examples.

The present application describes various technical characteristics and other advantages with reference to the examples and/or various embodiments disclosed herein. Those skilled in the art will appreciate that the technical features of a given embodiment may in fact be combined with features of another embodiment unless the inverse is explicitly mentioned or unless it is obvious that these features are incompatible or that this combination does not provide a solution to at least one of the technical problems mentioned in the present application. Furthermore, the technical characteristics described in one embodiment can be isolated from the other features of this mode unless the inverse is explicitly mentioned. Consequently, the present embodiments must be considered illustrative, but they can be modified in the range defined by the scope of the attached claims.

The following examples are intended to illustrate the above invention and should not be construed as to narrow its scope. One skilled in the art will readily recognize that the Examiners suggest many other ways in which the invention could be practiced. It should be understood that numerous variations and modifications may be made while remaining within the scope of the invention.

### EXAMPLES

### Catalysts/Precatalysts Synthesis

General Procedure.

All experiments were carried out using standard air-free methodology in an argon-filled glovebox, on a Schlenk line or in a Schlenk-type apparatus interfaced to a high-vacuum line (about 10⁻⁵ mbar). Grafting was performed in double Schlenk flask that allowed filtration through sintered frits. In this apparatus solvent can be redistilled on the grafted oxide side, and thus further washing can be carried out avoiding use of fresh solvent. Solvents were purified and dried according to standard procedures. The support Bi₂O₃—SiO₂ was prepared by co-precipitation of Bi(NO₃).5H₂O and silica in acetic acid. After calcination at about 500° C., this support was dehydroxylated at about 200° C. (noted Bi₂O₃—SiO₂) The precursors of Molybdenum were synthesized according to the literature: MoO₂Mes₂ (Mes=1,3,5 trimethylphenyl) (Kirsten, G.; Görls, H.; Seidel, W. Z. Für Anorg. Allg. Chem. 1998, 624 (2), 322-326); Mo(═N^{t}Bu)₂Cl₂(DME) (^{t}Bu tert-butyl). (Fox, H. H.; Yap, K. B.; Robbins, J.; Cai, S.; Schrock, R. R. Inorg. Chem. 1992, 31 (11), 2287-2289); Mo(═N-2,6-C₆H₃-ⁱPr₂)₂Cl₂(DME) (ⁱPr=iso-propyl); Fox, H. H.; Yap, K. B.; Robbins, J.; Cai, S.; Schrock, R. R. Inorg. Chem. 1992, 31 (11), 2287-2289); Mo(═N^{t}Bu)₂(CH₂^{t}Bu)₂; Mo(═N-2,6-C₆H₃-ⁱPr₂)₂ (CH₂^{t}Bu)₂ (Bryson, N.; Youinou, M. T.; Osborn, J. A. Organometallics 1991, 10 (9), 3389-3392); Mo(≡C^{t}Bu)(CH₂^{t}Bu)₃; (McCullough, L. G.; Schrock, R. R.; Dewan, J. C.; Murdzek, J. C. J. Am. Chem. Soc. 1985, 107 (21), 5987-5998); MoONp₃Cl. (Kress, J. R. M.; Russell, M. J. M.; Wesolek, M. G.; Osborn, J. A. J C Chem Commun 1980, 431-432); MoO(O^{t}Bu)₄. (Stoffelbach, F.; Saurenz, D.; Poli, R. Eur. J. Inorg. Chem. 2001, 2001 (10), 2699-2703 and Chisholm, M. H.; Folting, K.; Huffman, J. C.; Kirkpatrick, C. C. Inorg. Chem. 1984, 23 (8), 1021-1037); Bi(O^{t}Bu)₃. (Evans, W. J.; Hain, J. H.; Ziller, J. W. J. Chem. Soc. Chem. Commun. 1989, No. 21, 1628-1629).

Elemental analyses were performed at Mikroanalytisches Labor Pascher. Gas-phase analyses were performed on a Hewlett-Packard 5890 series II gas chromatograph equipped with a flame ionization detector and HP5 (30 m×0.32 mm) or KCl/Al₂O₃ (50 m×0.32 mm) column for t-butanol or isobutene determination, respectively. Diffuse reflectance infrared spectra were collected in a Nicolet 6700 FT-IR spectrophotometer in 4 cm⁻¹ resolutions. An air-tight IR cell with CaF₂ window was applied and the final spectra comprise 64 scans. Raman spectroscopy was performed on a Horiba Yvon LabRAM Aramis with a CCD-camera as a detector using a 50× objective, an 1800 gr/mm grating, a 100 μm slit and a 473 nm cobalt laser. The Raman spectra were collected on the samples sealed under Ar atmosphere, which were packed in a closed cell fitted with rubber O ring and a Quartz window. Solution NMR spectra were recorded on an Avance-300 Bruker spectrometer. All chemical shifts were measured relative to residual ¹H or ¹³C resonances in the deuterated solvent: C₆D₆, δ 7.16 ppm for ¹H, 128.06 ppm for ¹³C. ¹H and ¹³C solid-state NMR spectra were recorded on Brilker Avance-500 spectrometers with a conventional double-resonance 4 mm CP-MAS probe. The samples were introduced under argon in a zirconia rotor (4 mm), which was then tightly closed. In all experiments, the rotation frequency was set to 10 kHz. Chemical shifts were given with respect to TMS as external reference for ¹H and ¹³C NMR. Diffuse reflectance UV-vis spectra in the range 200-800 nm were taken on a Perkin Elmer λ1050 UV-Vis-NIR Spectrophotometer adapted with the Praying Mantis optical unit provided by Harrick. The spectrum for material 1b was recorded against BaSO₄ baseline. An airtight cell with quartz windows was used. The UV-vis spectra were processed with Microsoft Excel software, consisting of calculation of the Kubelka-Munk function, F(R_{∞}), which was extracted from the UV-vis DRS data. The edge energy (E_{g}) for allowed transitions was determined by finding the intercept of the straight line in the low-energy rise of a plot of [F(R_{inf})hν]^{1/n}, where n=0.5 for the direct allowed transition, vs hν, where hν is the incident photon energy. Geometry optimizations and calculations of thermochemical corrections.

All geometry optimizations were performed with the PBE GGAS10 DFT functional as implemented in PRIRODA 13 electronic structure code. All electron basis sets (λ1) of valence double-ζ plus polarization quality were used. All stationary molecular geometries were characterized by analytically calculated matrix of electronic energy second derivatives with respect to nuclear coordinates (Hessian). No imaginary frequencies were found for all reactants, products and intermediates. Only one imaginary frequency was detected for all transitions states. Possible relativistic effects (for molybdenum) were taken into account via the scalar Dyall Hamiltonian. The default, adaptively generated PRIRODA grid, corresponding to an accuracy of the exchange-correlation energy per atom (1×10⁻⁸ Hartree) was decreased by a factor of 100 for more accurate evaluation of the exchange-correlation energy term. Default values were used for the Self-Consistent-Field (SCF) convergence and the maximum gradient for geometry optimization criterion (1×10⁻⁴ au), whereas the maximum displacement geometry convergence criterion was decreased to 0.0018 au. Translational, rotational, and vibrational partition functions for thermal corrections to arrive at total Gibbs free energies were computed within the ideal-gas, rigid-rotor, and harmonic oscillator approximations. The temperature used in the calculations of thermochemical corrections was set to 298.15 K in all the cases. The energies were re-evaluated in Single-Point fashion at optimized geometries by means M06 functional as implemented in Gaussian 09 code. The all-electron def2-tzvpp basis sets of Ahlrichs were used on all main-group elements. On molybdenum atom the Stuttgart ECP was used with the corresponding valence def2-tzvpp basis set. The “Integral (grid=ultrafine)” option was used for evaluation of the exchange-correlation term. The default value for the SP SCF convergence was adopted.

Silica Model.

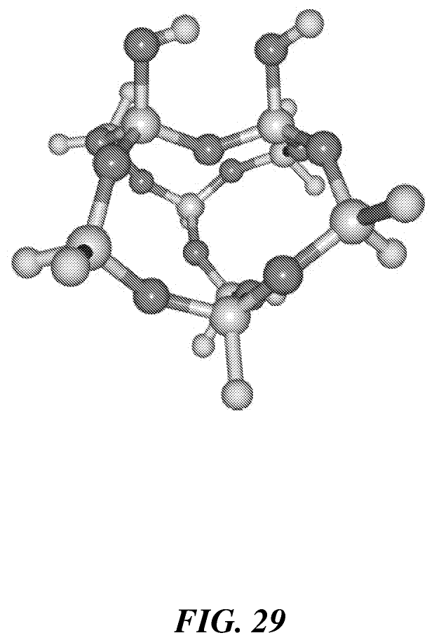

A relatively large cluster model cut out from the J cristobalite-based SiO₂ surface (model 001-4 in the paper) published by Rozanska et al (Table 1). has been chosen to simulate a silica surface, see FIG. 29. The spectra were acquired at ESRF, using beam-line BM23, at room temperature at the molybdenum K-edge, with a double crystal Si(111) monochromator detuned 70% to reduce the higher harmonics of the beam. The spectra were recorded in the transmission mode between 19.7 and 21.2 keV, every 0.3 eV in the edge area and every 1 eV for EXAFS. Four scans were collected for each sample. Each data set was collected simultaneously with a Mo foil reference (19999.5 eV), and was later aligned according to that reference (maximum of the first derivative of the first peak of the Mo foil). The Mo sample was packaged within an argon filled glovebox in a double air-tight sample holder equipped with kapton windows. The data analyses were carried out using the program “Athena” and the EXAFS fitting program “RoundMidnight”, from the “MAX” package, using spherical waves. The program FEFF8 was used to calculate theoretical files for phases and amplitudes based on model clusters of atoms. The scale factor, S₀²=0.68, was evaluated from the crystallized molecular complex Mo═(O)Ns₂(ONp)₂, characterized by XRD (almost square-based pyramid with an oxo in the apical position; 1.699 Å for M═O; 1.87 Å for M—O; 2.159 Å for M—C). This sample was studied diluted in BN and conditioned as a wafer. The refinements were carried out by fitting the structural parameters Nᵢ, Rᵢ, σᵢ and the energy shift, ΔE₀ (the same for all shells). The fit residue, ρ (%), was calculated by the following formula:

$$ρ = \frac{{\sum }_{k} {[ {k}^{3} {χ}_{exp} ( k ) - {k}^{3} {χ}_{cal} ( k ) ]}^{2}}{{\sum }_{k} {[ {k}^{3} {χ}_{exp} ( k ) ]}^{2}} * 100$$

The minimization of the quality factor, (Δχ)²/ν, (ν: number of degrees of freedom in the signal), was considered in order to control the number of variable parameters in the fit, as recommended by the Standards and Criteria Committee of the International XAFS Society.

Comparison of the reported bond distances for Mo═O and Mo═O bonds in selected bis-
oxo siloxy molybdenum molecular complexes

| Example | Observed distance (Å) | Reference |
|---|---|---|
| [(^{T}Bu)₂Si(O)₂MoO₂]₂.py | 1.694 to 1.708 Å for Mo═O and | Gosink, H.-G., Roesky, H. |
| | 1871 to 1.910 Å for Mo—OSi; | W. Nohemeyer, M., |
| | with Mo—N at 2.305(3) Å | Schmidt, H.-G., Freire- |
| | | Erdbrügger; C. & Sheldrick, |
| | | (G. M. Chem. Ber. 126, 279- |
| | | 283(1993). |
| [MoO₂{O(CH₂)₂S(CH₂)₂OH}(OSiPBuPH₂)] | 1.702 to 1.708 Å for Mo═O and | Ma, X., Yang, Z. Ringe, A. & |
| | 1.891(2) for Mo—OSi, with | Magull, J. Z. Anorg, Allg. |
| | Mo—OCH₂— at 1.990(2) Å and | Chem. 633, 1320-1322 |
| | Mo—O(H)CH₂— at 2.303(2) Å | (2007). |
| [(c-C₅H₉)₇Si₇(OTMS)MoO₂(NC₅H₅)]₂ | 1.687 to 1.701 Å for Mo═O and | Feher, F. J.; Rahimian, K.; |
| | 1.896 to 1.911 Å for Mo—OSi; | Budzichowski, T. A. & |
| | with Mo—N at 2.283(9) Å | Ziller; J. W. Organometallics |
| | | 14, 3920-3926(1995) |
| [MoO₂(OSiPh₃)₂(PPh₃)ₓ], with x = 0 or 3 | 1.663 to 1.699 Å for Mo—O and | Huang, M. Se DeKock, C. W . |
| | 1.810 to 1.928 Å for Mo—O, the | Inorg. Chem 32, 2287-2291 |
| | shortest bond lengths being | (1993). |
| | observed for the complex without | |
| | coordinated phosphine | |

### Example 1

Preparation of SiO₂₋₂₀₀.

Aerosil silica from Evonik with a specific area of about 200 m².g−1, was partly dehydroxylated at about 200° C. under high vacuum (about 10⁻⁵ Torr) for about 15 h to give a white solid having a specific surface area of about 190 m².g⁻¹ and containing about 2.4 OH.nm⁻² and about 0.8 mmol OH per g of SiO₂₋₂₀₀.

### Example 2

Preparation of Bi₂O₃ doped SiO₂₋₂₀₀ using SOMC procedures (Noted Bi₂O₃—SiO₂₋₂₀₀).

SiO₂₋₂₀₀ (15 g) was reacted overnight as a suspension in a stirring toluene (90 mL) solution of Bi(O^{t}Bu)₃ (2.92 g, 6.8 mmol) in the glovebox at 25° C. After filtration and washing of the obtained material with toluene and pentane, the resulting white powder was heated under vacuum (10⁻⁵ Torr) at 80° C. for 16 h then at 500° C. for another 16 h under a stream of dry air. The obtained white powder was then rehydrated at room temperature following by heating at 100° C. for 8 h then dehydroxylated at 200° C. under vacuum (10⁻⁵ Torr).

### Example 3

Standard procedure I for the preparation of MoO₂Mes₂/SiO₂₋₂₀₀—as illustrated graphically in scheme 1

MoO₂Mes₂ was added as a solid portion to a stirring suspension of SiO₂₋₍₂₀₀₎ (3 g) in toluene (10 mL) at 25° C. in the glove box. When the solution starts to become yellow the excess MoO₂Mes₂ was filtered off. After filtration, the solid MoO₂Mes₂/SiO₂₋₂₀₀ was washed three times with toluene. The resulting green powder was heated at 80° C. under vacuum (10⁻⁵ Torr) overnight to give a bipodal [(≡SiO)₂MoO₂] specie (65%). Elemental Analysis % Mo=5.20 wt %, % C=2.07 wt %.

### Bisoxo Tetiobutoxy

### Standard Procedure II for the Preparation of Bipodal Mo(═NR)₂(X)₂/SiO₂₋₂₀₀, Mo(═NR)₂(X)₂/SiO₂—Bi₂O₃₋₂₀₀ (X=Alkyl, Aryl) as Illustrated Graphically in Scheme 2

### Example 4

### Example of Mo(═N-(2,6-C₆H₃-ⁱPr₂)₂(CH₂tBu)₂/SiO₂₋₂₀₀

Mo(═N-(2,6-C₆H₃-ⁱPr₂)₂(CH₂tBu)₂ (0.148 g, 0.251 mmol) was dissolve in 5 mL of dry pentane and was slowly added to the slurry of SiO₂₋₂₀₀ (1.0 g) in pentane (ca. 7 mL) and stirred at room temperature for 12 h. The solvent was then removed by filtration and solid residue was thoroughly washed with pentane (5×5 mL) followed by drying under dynamic vacuum for 2 h. The light orange solid obtained was further heated at 80° C. (under dynamic vacuum; <10⁻⁵ torr) (16 h) to yield the bipodal supported complex Mo(═N-(2,6-C₆H₃-ⁱPr₂)₂(CH₂tBu)₂/SiO₂₋₂₀₀, Elemental analysis % Mo, 1.55 wt %; % C, 4.69 wt %; % N, 0.47 wt %. ¹H MAS NMR (500 MHz) δ 6.9, 3.1 and 1.0 ppm. ¹³C CP MAS NMR (125 MHz) δ 152.0, 141.0, 123.0, 120.0, 26.8 and 21.0 ppm.

### Example 5

### Example of Mo(═N^{t}Bu)₂(CH₂tBu)₂/SiO₂₋₂₀₀

Mo(═N^{t}Bu)₂(CH₂tBu)₂ (0.18 g, 0.473 mmol) was dissolve in 5 mL of dry pentane and was slowly added to the slurry of SiO₂₋₂₀₀ (1.0 g) in pentane (ca. 7 mL) and stirred at room temperature for 12 h. The solvent was then removed by filtration and solid residue was thoroughly washed with pentane (5×5 mL) followed by drying under dynamic vacuum for 2 h. The light gray-brown solid obtained was further degassed at 80° C. (under dynamic vacuum; <10⁻⁵ torr) for another 16 h to yield material Mo(═N^{t}Bu)₂(CH₂tBu)₂/SiO₂₋₂₀₀. Elemental analysis % Mo, 2.60 wt %; % C, 2.88 wt %; % N, 0.66 wt %. ¹H MAS NMR (500 MHz) δ 1.3 ppm. ¹³C CP MAS NMR (125 MHz) δ 69.0, 51.0, 43.0 and 30.0 ppm. Published result Barman, S.; Merle, N.; Minenkov, Y.; De Mallmann, A.; Samantaray, M. K.; Le Quéméner, F.; Szeto, K. C.; Abou-Hamad, E.; Cavallo, L.; Taoufik, M.; Basset, J.-M. Organometallics 2017, 36 (8), 1550-1556.

### Example 6

### Example of Mo(═N-(2,6-C₆H₃-ⁱPr₂)₂(CH₂tBu)₂/Bi₂O₃—SiO₂₋₂₀₀

Mo(═N-(2,6-C₆H₃-ⁱPr₂)₂Np₂ (0.513 g, 0.87 mmol) was dissolved in 5 mL of dry pentane and was slowly added to the slurry of Bi₂O₃—SiO₂₋₂₀₀ (2.5 g) in pentane (ca. 7 mL) and stirred at room temperature for 12 h. The solvent was then removed by filtration and solid residue was thoroughly washed with pentane (5×5 mL) followed by drying under dynamic vacuum for 2 h. The orange solid obtained was further heated at 80° C. (under dynamic vacuum; <10⁻⁵ torr) (16 h) to yield material Mo(═N-(2,6-C₆H₃-ⁱPr₂)₂(CH₂tBu)₂/Bi₂O₃—SiO₂₋₂₀₀. Elemental analysis % Bi=12.5 wt %; % Mo, 1.49 wt %; % C, 4.38 wt %; % N, 0.53 wt %. ¹H MAS NMR (500 MHz) δ 7, 3.5 and 1.1 ppm. ¹³C CP MAS NMR (125 MHz) δ 153.0, 143.0, 124.0, 121.0, 28.0 and 21.0 ppm.

### Example 7

### Example of Mo(═N^{t}Bu)₂(CH₂tBu)₂/Bi₂O₃—SiO₂₋₂₀₀

Mo(═N^{t}Bu)₂(CH₂tBu)₂ (0.513 g, 0.87 mmol) was dissolve in 5 mL of dry pentane and was slowly added to the slurry of Bi₂O₃—SiO₂₋₂₀₀ (2.5 g) in pentane (ca. 7 mL) and stirred at room temperature for 12 h. The solvent was then removed by filtration and solid residue was thoroughly washed with pentane (5×5 mL) followed by drying under dynamic vacuum for 2 h. The light orange solid obtained was further heated at 80° C. (under dynamic vacuum; <10⁻⁵ torr) (16 h) to yield material Mo(═N^{t}Bu)₂(CH₂tBu)₂/SiO₂—Bi₂O₃₋₂₀₀. Elemental analysis % Bi=25 wt %; %1,5 Mo, ¹H MAS NMR (500 MHz) δ 1.3 ppm. ¹³C CP MAS NMR (125 MHz) δ 69.0, 51.0, 43.0 and 30.0 ppm.

### Standard Procedure III for the Preparation of Bipodal Mo(≡C^{t}Bu)(CH₂^{t}Bu)₃/Bi₂O₃—SiO₂₋₂₀₀ as Illustrated Graphically in Scheme 3

### Example 8

### Example of Mo(≡C^{t}Bu)(CH₂^{t}Bu)/Bi₂O₃—SO₂₋₂₀₀

Mo(≡C^{t}Bu)(CH₂^{t}Bu)₃ (0.345 g, 0.91 mmol) was dissolve in 5 mL of dry pentane and was slowly added to the slurry of Bi₂O₃—SiO₂₋₂₀₀ (2.6 g, around ˜1.9 mmol —OH) in pentane (ca. 7 mL) and stirred at room temperature for 12 h. The solvent was then removed by filtration and solid residue was thoroughly washed with pentane (5×5 mL) followed by drying under dynamic vacuum for 2 h giving a yellow material Mo(≡C^{t}Bu)(CH₂^{t}Bu)₃/Bi₂O₃—SiO₂₋₂₀₀ Elemental analysis % Bi=12.5 wt %; % Mo, 1.89 wt %; % C, 2.50 wt %. ¹H MAS NMR (500 MHz) δ 1.0 ppm. ¹³C CP MAS NMR (125 MHz) δ 89, 54, 31 and 28 ppm.

### Standard Procedure IV for the Preparation of Mo(═O)(CH₂^{t}Bu)₃Cl/SiO₂₋₂₀₀ and Mo(═O)Np₃Cl/Bi₂O₃—SiO₂₋₂₀₀ as Illustrated Graphically in Scheme 4

### Example 9

A mixture of finely ground Mo(═O)(CH₂^{t}Bu)₃Cl (120 mg, 0.33 mmol) and SiO₂₋₂₀₀ (1 g) were stirred at 25° C. under dynamic vacuum for 4 h, whereas all volatile compounds were condensed into a cold trap. Pentane was then added and the solid was washed 5 times. The resulting white powder was heated under vacuum (10⁻⁵ Torr) at 80° C. for 16 h. Analysis by infrared spectroscopy of the condensed volatiles indicated the formation of 218 μmol of HCl during the grafting. Elemental anal.: % Mo=2.38 wt %, % C=1.56 wt %, ¹H MAS NMR (500 MHz) δ 2.6, 1.1 ppm. ¹³C CP MAS NMR (125 MHz) δ 86.7, 35.4, and 30.6 ppm.

### Example 10

A mixture of finely ground Mo(═O)Np₃Cl (0.350 g, 0.97 mmol) and Bi₂O₃—SiO₂₋₂₀₀ (2.5 g) were stirred at 25° C. under dynamic vacuum for 4 h, whereas all volatile compounds were condensed into a cold trap. Pentane was then added and the solid was washed 5 times. The resulting light brown powder was heated under vacuum (10⁻⁵ Torr) at 80° C. for 16 h. Analysis by infrared spectroscopy of the condensed volatiles indicated the formation of 650 μmol of HCl during the grafting. Elemental analysis.: % Bi=12.5 wt %; % Mo=2.07 wt %, % C=1.58 wt %, ¹H MAS NMR (500 MHz) δ 1.2 ppm. ¹³C CP MAS NMR (125 MHz) δ 86.7, 36, and 32 ppm.

### Standard Procedure V for the Preparation of Mo(═O)₂/SiO₂₋₂₀₀ as Illustrated Graphically in Scheme 5

### Example 11

In a glovebox a double Schlenk was charged with [(O═)Mo(O^{t}Bu)₄] (about 912 mg, about 2.25 mmol) and partially dehydroxylated silica at about 200° C. SiO₂₋₂₀₀ (2.5 g). After evacuation of the double Schlenk to about 10⁻⁵ mbar, pentane was distilled over the complex, the obtained solution was then transferred onto the silica. After about 3 h reaction at about room temperature, the supernatant was filtered off and the solid washed five times with pentane by reusing it doing distillation-condensation cycles. Evaporation of the volatiles and qualitative analysis of them revealed the presence of tert-butanol. Material (≡Si—O)₂Mo(═O)(OtBu)₂ was obtained as a slightly yellow powder after drying under vacuum (10⁻⁵ mbar).

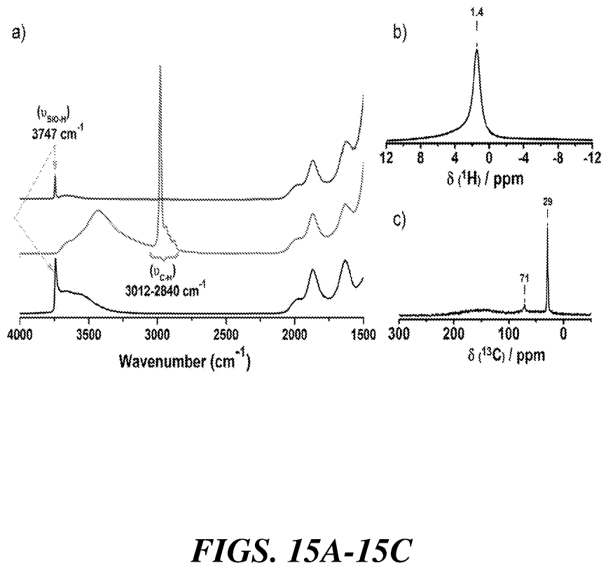

Diffuse reflectance infrared Fourier transform (DRIFT) spectroscopy revealed consumption of the isolated silanols of SiO₂₋₍₂₀₀₎ (ν(SiO—H)) at 3,747 cm⁻¹ (FIG. 15A). New peaks that corresponded to ν (C—H) of the tert-butoxy fragments also appeared. The broad adsorption from 3,700 to 3,100 cm⁻¹ was related to H-bonding interactions between the tert-butoxy ligands and the remaining surface silanols. Qualitative gas chromatography (GC) analysis of the filtrate after washing indicated the presence of ^{t}BuOH, which originated from silanolysis of the tert-butoxy Mo fragments. Elemental analysis of the resulting material indicated Mo and C contents of about 3.47 and 3.64% wt, respectively. This C/Mo molar ratio (i.e., about 8.4) was close to the expected value of 8 for (≡Si—O)₂Mo(═O)(OtBu)₂.

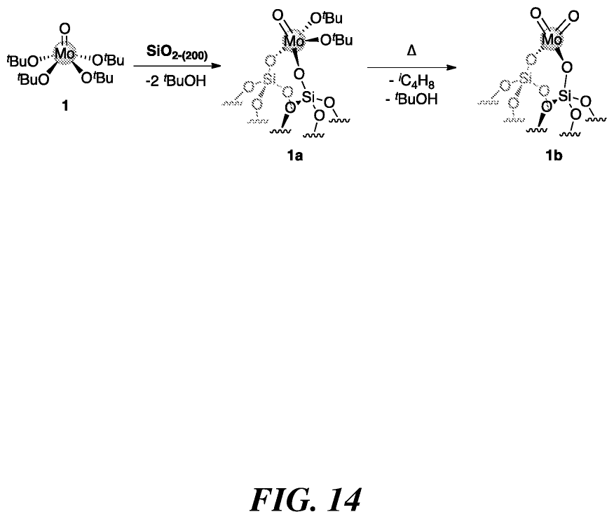

The ¹H magic angle spinning (MAS) and ¹³C cross-polarization magic angle spinning (CP-MAS) nuclear magnetic resonance (NMR) (FIGS. 15B-15C) data confirmed the presence of Mo tert-butoxy fragments based on a ¹H peak at about 1.4 ppm and ¹³C peaks at about 29 and 71 ppm. The results obtained from different spectroscopic methods as well as the elemental analysis suggested that the reaction of [O═Mo(O^{t}Bu)₄] with SiO₂₋₍₂₀₀₎ proceeded by Mo—O cleavage along with tBuOH release, leading to the bipodal surface species [(≡Si—O—)₂Mo(═O)(O^{t}Bu)₂] (FIG. 14).

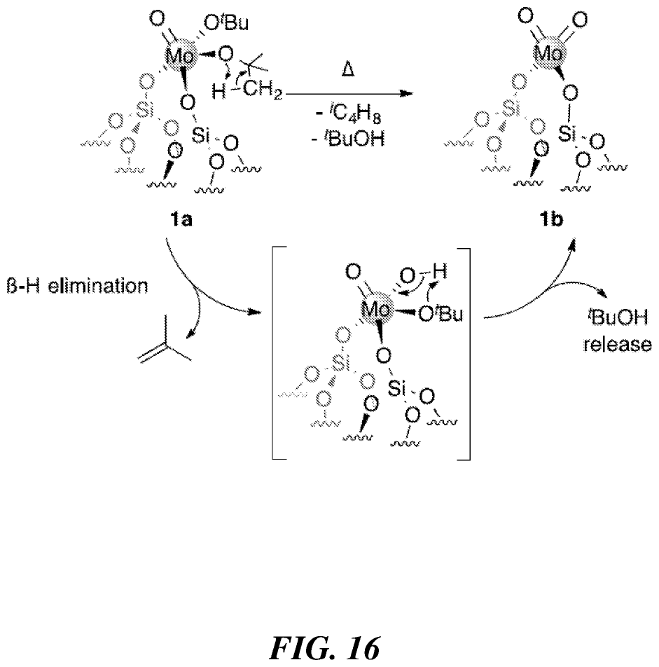

Thermolysis of supported complex [(≡Si—O—)₂Mo(═O)(O^{t}Bu)₂] at about 250° C. (for about 2 h) under high vacuum (about 10⁻⁵ mbar) triggered elimination of the H atom from one of the methyl groups of [(≡Si—O—)₂Mo(═O)(O^{t}Bu)₂] to the β oxygen atom to form the bipodal Mo oxohydroxotert-butoxide [(≡Si—O—)₂Mo(═O)(OH)(O^{t}Bu)] intermediate with the release of about 0.85 equivalent of isobutene/grafted Mo (GC) (FIG. 16). Further heating of this intermediate led to α-H abstraction, which quantitatively released ^{t}BuOH (FIG. 16), and formation of a Molybdenum species that was characterized as the silica-supported single-site catalyst (≡Si—O—)₂Mo(═O)₂.

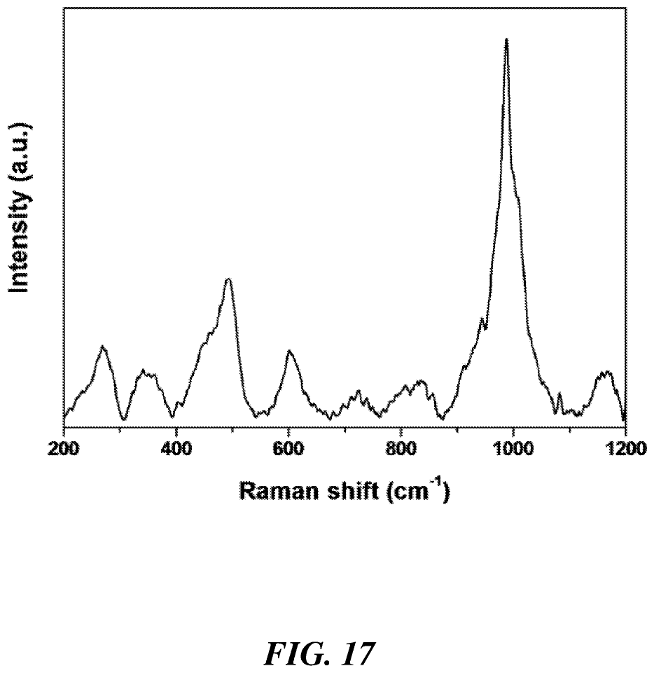

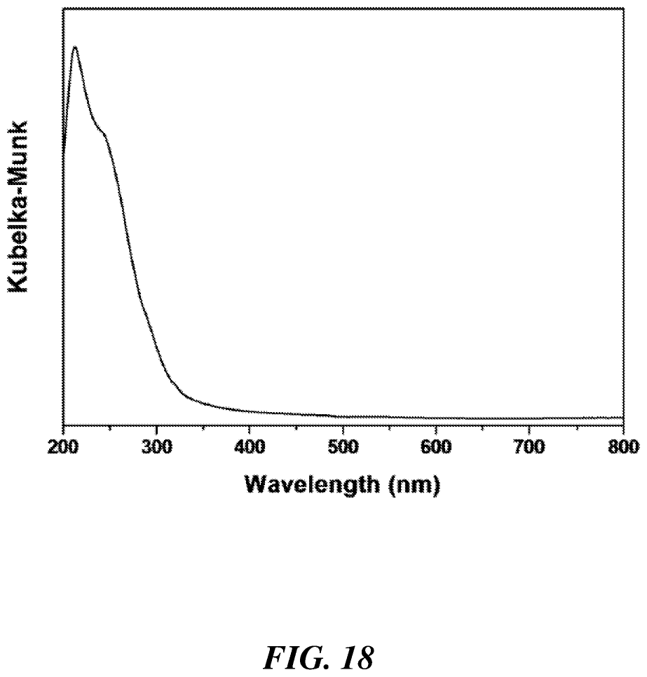

The DRIFT analysis of ((≡Si—O)₂Mo(═O)₂) revealed the disappearance of the alkyl vibrational bands (about 3,000-2,800 cm⁻¹), which was accompanied by the re-appearance of isolated silanol groups at about 3,747 cm⁻¹ (FIG. 15A). The Raman spectrum of ((≡Si—O)₂Mo(═O)₂) (FIG. 17) contained broad Raman features at about 400-500 and 800-900 cm⁻¹ as well as a smaller feature at about 610 cm⁻¹, corresponding to the various vibrational modes of the siloxane bridges. Importantly, a strong band that was centered at about 986 cm⁻¹ was observed, which was attributed to a combination of the stretching vibrations of terminally bound bis-oxo ligands in ((≡Si—O)₂Mo(═O)₂). Furthermore, no features attributed to Mo—O—Mo were observed at 270, 720, or 805 cm⁻¹, suggesting the absence of oligomeric Mo species. The diffuse reflectance ultraviolet-visible (UV-Vis) spectrum of ((≡Si—O)₂Mo(═O)₂) (FIG. 18) indicated characteristic ligand-to-metal charge transfer centers at about 212 and 240 nm, corresponding to a band gap value (E_{g}) of about 3.9 eV. Although the reported band gap value for isolated, perfectly tetrahedral MoO₄ units (Na₂MoO₄) was about 4.7 eV, a distortion of the tetrahedral geometry may lower the band gap value (E_{g} of Al₂(MoO₄)₃=4.2 eV).

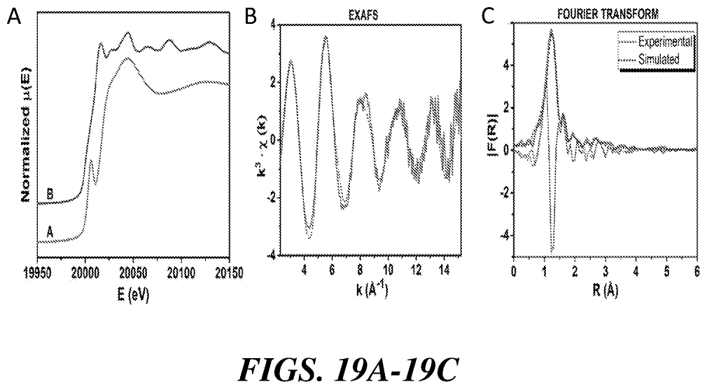

The structure of the supported species ((≡Si—O)₂Mo(═O)₂) was studied using X-ray absorption spectroscopy (XAS), X-ray absorption near edge structure (XANES) spectroscopy and extended X-ray absorption fine structure (EXAFS) spectroscopy (FIGS. 19A-19C and Table 1). The XAS data suggested that this catalyst can be described by isolated (≡Si—O—)₂Mo(═O)₂ units with a deformed tetrahedral structure due to the heterogeneity of the silica support and the variation in the O—Mo—O angle. The XANES analysis of ((≡Si—O)₂Mo(═O)₂) (FIG. 19A) indicated an intense pre-edge peak at about 20,006.1±0.3 eV, which was characteristic of a dipole-forbidden 1s→4d transition. Although this transition is forbidden, the formation of molecular orbitals mixing Mo 4d and 5p orbitals with orbitals of the ligands allowed the appearance of a pre-edge peak when Mo(VI) had a tetrahedral or quasi-tetrahedral symmetry. In contrast, the pre-edge signal was very weak in complexes with octahedral or pseudo-octahedral symmetry. The Mo K-edge corresponded to the dipole-allowed 1s→5p transition, which was typically sensitive to both the oxidation state of Mo and the bond covalence. The transition was positioned at 20,017±1 eV (the maximum of the first derivative of the edge), and the energy at the half-step height was 20,014±1 eV, indicating a +VI formal oxidation state for Mo. This result confirmed that the molybdenum in ((≡Si—O)₂Mo(═O)₂) was Mo(VI) with tetrahedral or nearly tetrahedral symmetry. Fitting of the EXAFS signal (Table 1) suggested the following coordination sphere for Mo: (i) ca. two oxygen atoms at 1.705(10) Å, which were assigned to two Mo═O oxoligands, and (ii) ca. two oxygens at 1.870(15) Å, which were assigned to the “surface siloxide ligands”. These bond lengths for the Mo═O and Mo—O bonds were in the range of bond lengths observed for soluble bis-oxo-siloxy Mo molecular complexes. Similar parameters were obtained when fitting the k².χ(k) spectrum. The fit can be improved by adding a layer of further back-scatterers with ca. one oxygen and two silicon atoms at 2.39(4) and 3.27(5) Å, respectively, due to a surface oxygen atom from a siloxane bridge of the silica support and the silicon atoms of the surface siloxide ligands. Therefore, this EXAFS study was in agreement with the structure for the catalyst as (≡Si—O—)₂Mo(═O)₂.

TABLE 1
EXAFS (≡Si—O—)₂Mo(═O)₂.*

| | | Distance | σ² |
|---|---|---|---|
| Type of neighbor | No. of neighbors | (Å) | (Å²) |
| Mo═O | 2.1(3) | 1.705(10) | 0.0019(5) |
| Mo—OSi≡ | 1.9^{b} | 1.870(15) | 0.0032(11) |
| Mo—O(Si≡)₂ | 0.7(5) | 2.39(4) | 0.006(5) |
| Mo—O—Si≡ | 1.9^{b} | 3.27(5) | 0.014(12) |

The errors generated by the EXAFS fitting program “RoundMidnight” are indicated in parentheses. *Δk: [12.4-15.2 Å⁻¹] − ΔR: [0.4-3.4 Å] ([0.4-2.0 Å], when considering only the first coordination sphere); S0² = 0.68; ΔE₀ = 4.5 ± 1.2 eV (the same for all shells); Fit residue: ρ = 6.7%; Quality factor: (Δχ)²/v = 2.96, with v = 15/26 ([(Δχ)²/v]1= 3.46 with v = 9/15, considering only the first coordination sphere: ═O and —O).
^{b}Shell constrained to a parameter above (2N(═O) + N(O) = 6; N(O) = N(Si)).

### Catalytic Studies

All gases were purchased from Abdul Hashim gases (AHG). Grade 5.0 helium, nitrogen and oxygen were used here, whereas propene (C₃H₆) and cis-2-butene were grade 4.0 and 3.5. All inert gases were further purified using triple filters purchased from Agilent to remove traces of water, O₂, and hydrocarbons. C₃H₆ was further purified using molecular sieve 3 Å and a copper-based catalyst to remove water and O₂, respectively. All catalytic tests were conducted in gas-phase using a fixed-bed isothermal flow reactor purchased from Process Integral Development and Engineering Technology^{@} (PI&DEng.& Tech.). The flow of all gases was controlled using calibrated Bronkhorstv^{@} mass flow controllers. First, 75 to 200 mg of catalyst was loaded in a stainless steel reactor (length 30 cm and I.D. 9 mm) inside a glove box under inert atmosphere. To avoid any exposure of the catalyst to air, both ends of the tube were connected to a two positions 4-way valve that can be manually switched between two positions allowing the possibility of by-passing the reactor. Later, a gas mixture containing 6-8 v/v % olefins, 2.5-10 v/v % O₂, 12.5 v/v % N₂, in He (Bal.) was introduced into the reactor using a total flow rate of 32 mL/min @ NTP (16 mL/min @ NTP in case of cis-2-pentene). Propylene and butene-2 were used in the gas phase. With cis-2-pentene which was mostly liquid at room temperature was introduced into the catalytic system using a saturator bubbled with He (its flow was controlled by a calibrated Bronkhorst mass flow controllers). The control of the flow rate of cis-2-pentene was achieved by controlling the temperature of the liquid in the saturator chamber and the He flow rate. The exact molar fraction of cis-2-pentene in the total feed was evaluated by previously calibrating the GC system (through manual injection) using highly pure (analytical grade) liquid sample of cis-2-pentene. Finally, the reactor was heated to the required temperature (about 350-450° C.) and the reaction was studied under steady-state conditions. On-line gas analysis of the products was performed on a Varian 450 GC gas chromatograph. A sample from the reactor outlet stream was automatically injected on three parallel channels referred to here as channel A, channel B and channel C. In Channel A, the sample (1 mL @ STP) was injected on a set of three packed columns, “Hayesep” ^{@} Q (CP81073), “Hayesep” T (CP81072), and “Molsieve” ^{@} 13X (CP81073) connected in series. A set of 10-way and 6-way Valco ^{@} valves were used to allow automatic injection of the sample, back-flushing of Hayesep T, and by-passing of Molsieve 13X columns. This channel was equipped with a TCD detector (He as reference gas) and used to monitor the amount of CO and CO₂, O₂ and N₂. Channel B uses HP-AIJKCL column. This channel was equipped with a FID detector and used to monitor hydrocarbons. Channel C uses HP-PLOT U for the studies using propylene as substrate whereas or HP-PLOT Q was used for the studies using other olefin substrates. This channel was equipped with a FID detector and used to monitor oxygenates and some of the selected hydrocarbons.

Conversion X (expressed in %) was calculated using the following formula

$$X = \frac{( {x}_{N 2} - {x}_{3} )}{{x}_{N 2}} \times \frac{{x}_{N 2}^{°}}{{x}_{3}^{°}}$$

And the carbon selectivity of carbonaceous reaction product i was assessed as follows

$${S}_{i} = \frac{( {n}_{i} {x}_{i} )}{Σ {n}_{i} {x}_{i}}$$

Here, x^{∘hd 3} and x₃ are the initial molar fractions of butene-2, propylene or pentene at the reactor inlet and outlet respectively. x^{∘}_{N2} and x_{N2} are the molar fraction of N₂ at the inlet and the outlet of the reactor resp. xᵢ is the molar fraction of carbonaceous product i at the reactor outlet, whereas nᵢ is the number of carbons in hydrocarbon i. In most of the studies, the carbon mass balance was found in the range of 94-99%. Higher values of mass balance could not be obtained because of the number of compounds analyzed.

### Conversion of Propylene or Butene by Molecular Oxygen Using a Single-Site Olefin Metathesis Catalyst in Presence or Absence of Ammonia

### Example 12

The bipodal catalyst Mo(═N^{t}Bu)₂(CH₂tBu)₂/SiO₂₋₂₀₀ is prepared according to the method disclosed in detail in the above catalysis standard procedure II.

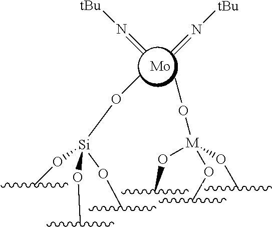

Table 2 shows the catalytic metathetic-oxidation and/or ammoxidation results obtained with this catalyst in function of the volume fraction of oxygen and ammonia.

TABLE 2

| | Feed ratio | % Conv | Selectivity (%) | | | | | |
|---|---|---|---|---|---|---|---|---|
| Entry | C₃ = /NH₃/O₂/N₂/He | C₃ = | Ethanal | Propanal | Propenal | HCN | CH₃CN | ACN |
| 1 | 7.5/0/10/12.5/70 | 5-6 | 21 | 1.5-2 | 8-9 | 0 | 0 | 0 |
| 2 | 7.5/7.5/7.5/12.5/65 | 4-5.5 | 38-40 | 0.0 | 1-2 | 3-5 | 23-25 | 12-14 |

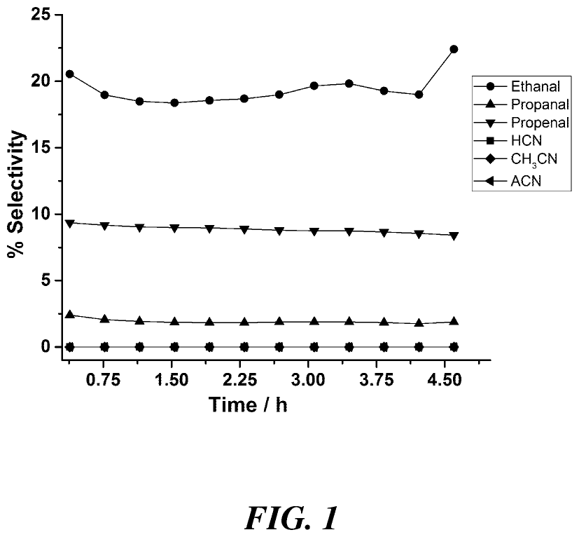

Reaction conditions: T = 380° C.;
weight of catalysts = 25 mg;
Total flow of gas mixtures: 32 mL/min;
Gas hourly space velocity (GHSV) of 768000 h⁻¹;
W/F: 0.0468 g · s · mL⁻¹; acrylonitrile is noted in FIG. 1 and Table 2 by ACN.
Other minor products with selectivity <1-2% and over oxidation products CO₂ and CO are not presented in the table 2.

FIG. 1 depicts the selectivity vs time on stream plot of propene metathetic-oxidation under select conditions over the catalyst (reaction conditions: T=380° C.; Feed ratio C₃═/NH₃/O₂/N₂/He=7.5/0/10/12.5/70), according to one or more embodiments of the present disclosure.

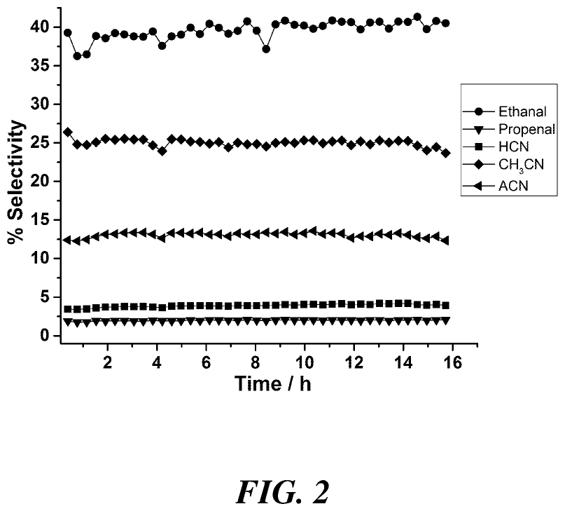

FIG. 2 depicts the selectivity vs time on stream plot of propene ammoxidation under selected condition over catalyst (reaction conditions: T=380° C.; Feed ratio: C₃═/NH₃/O₂/N₂/He=7.5/7.5/7.5/12.5/65), according to one or more embodiments of the present disclosure.

### Example 13

The catalyst MoO₂Mes₂/SiO₂₋₂₀₀ is prepared according to the method disclosed in detail in the above standard procedure I.

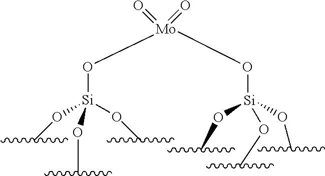

Table 3 gives the catalytic metathetic-oxidation and/or ammoxidation results obtained with (≡Si—O—)₂Mo(═O)₂ catalyst.

TABLE 3

| | Feed ratio | % Conv | % Selectivity | | | | | |
|---|---|---|---|---|---|---|---|---|
| Entry | C₃ = /NH₃/O₂/N₂/He | C₃ = | Ethanal | Propanal | Propenal | HCN | CH₃CN | ACN |
| 3 | 7.5/0/7.5/12.5/72.5 | 3-4 | 28 | 21 | 10 | 0 | 0 | 0 |
| 4 | 7.5/0/5/12.5/75 | 2-3 | 23 | 33 | 7 | 0 | 0 | 0 |
| 5 | 7.5/0/10/12.5/70 | 2-3 | 27-31 | 34 | 8-9 | 0 | 0 | 0 |
| 6 | 7.5/1.875/7.5/12.5/70.6 | 4-6 | 34 | 2 | 2 | 3 | 27 | 6 |
| 7 | 7.5/1.875/10/12.5/68.1 | 4-6.5 | 37 | 2.6 | 2.6 | 2.4 | 23 | 5 |
| 8 | 7.5/3.75/10/12.5/66.3 | 5-6 | 36 | 1 | 2 | 3 | 29 | 5 |
| 9 | 7.5/7.5/10/12.5/62.5 | 3-5 | 30 | 0 | 1.4 | 3-4 | 39 | 5 |

Reaction conditions: T = 380° C.;
Cat. Weight of Catalyst: 30 mg;
Total flow: 32 mL/min;
Gas hourly space velocity (GHSV) of 64000 h⁻¹;
W/F: 0.056 g · s · mL⁻¹;
(ACN = acrylonitrile).
Other minor products with selectivity <1-2% and over oxidation products CO₂ and CO are not presented in the table.

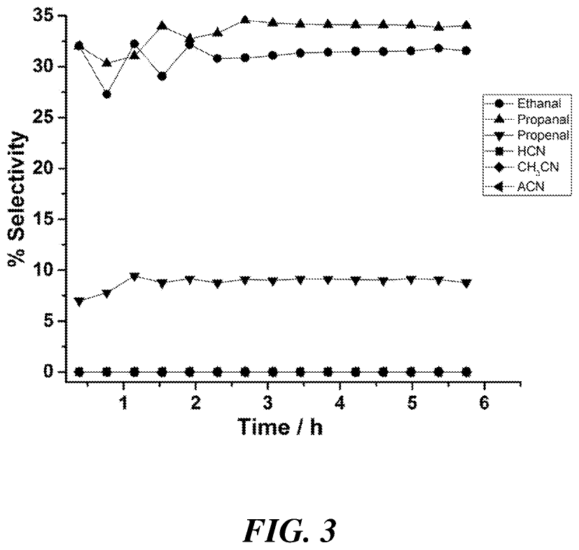

FIG. 3 depicts the selectivity vs time on stream plot of propene metathetic-oxidation under selected condition over MoO₂Mes₂/SiO₂₋₂₀₀ (reaction conditions: T=380° C.; Feed ratio: C₃═/NH₃/O₂/N₂/He=7.5/0/10/12.5/70), according to one or more embodiments of the present disclosure.

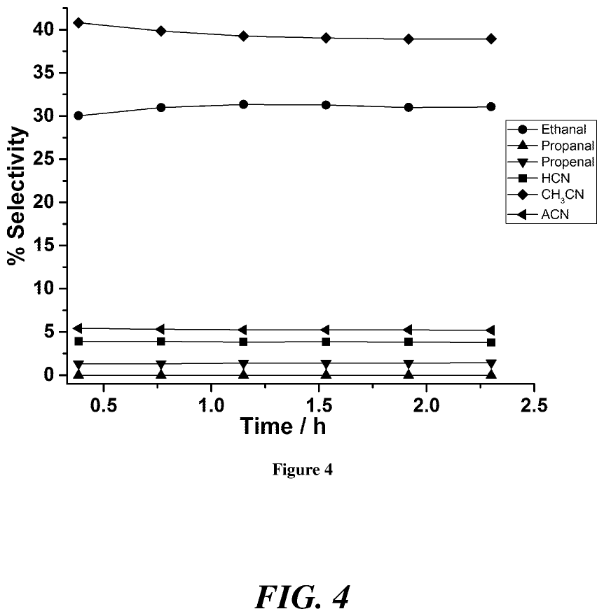

FIG. 4 depicts the selectivity vs time on stream plot of propene ammoxidation under selected condition over MoO₂Mes₂/SiO₂₋₂₀₀ (Feed ratio: C₃═/NH₃/O₂/N₂/He=7.57.5/10/12.5/62.5), according to one or more embodiments of the present disclosure.

### Example 14

The catalyst Mo(═N-(2,6-C₆H₃-ⁱPr₂)₂(CH₂tBu)₂/Bi₂O₃—SiO₂₋₂₀₀ Bi=10.0% wt, Mo=1.49% wt is prepared according to the method disclosed in detail in the above standard procedure II with different supports (Bismuth doped silica.

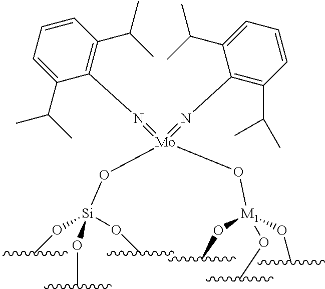

Table 4 gives the catalytic metathetic-oxidation and/or ammoxidation results obtained with Mo(═N-(2,6-C₆H₃-ⁱPr₂)₂(CH₂tBu)₂/Bi₂O₃—SiO₂₋₂₀₀ catalyst.

TABLE 4

| | Feed ratio | % Conv | % Selectivity | | | | |
|---|---|---|---|---|---|---|---|
| Entry | C₃ = /NH₃/O₂/N₂/He | C₃ = | Ethanal | Propenal | HCN | CH₃CN | ACN |
| 10 | 7.5/0/5/12.5/75 | 3-4 | 38-42 | 30-34 | 0 | 0 | 0 |
| 11 | 7.5/0/7.5/12.5/72.5 | 2.5-3 | 30-34 | 35 | 0 | 0 | 0 |
| 12 | 7.5/0/10/12.5/70 | 3-4 | 36-43 | 30-40 | 0 | 0 | 0 |
| 13 | 7.5/1.875/7.5/12.5/70.6 | 4-4.5 | 35-38 | 9-10 | 3 | 12 | 13 |
| 14 | 7.5/1.875/10/12.5/68.1 | 4-5 | 36 | 9 | 3 | 12 | 12 |
| 15 | 7.5/3.75/10/12.5/66.3 | 4-6 | 32 | 6 | 4 | 15 | 13 |
| 16 | 7.5/7.5/10/12.5/62.5 | 4-5 | 37 | 7 | 5 | 10 | 20 |
| 17 | 7.5/7.5/2.5/12.5/70 | 3-6 | 30-45 | 10-12 | 5 | 10-11 | 24-30 |

Reaction conditions: T = 380° C.;
weight of catalyst: 30 mg;
Total flow: 32 mL/min;
Gas hourly space velocity (GHSV) of 64000 h⁻¹;
W/F: 0.056 g · s · mL⁻¹;
ACN = acrylonitrile.
Other minor products with selectivity <1-2% and over oxidation products CO₂ and CO are not presented in the table.

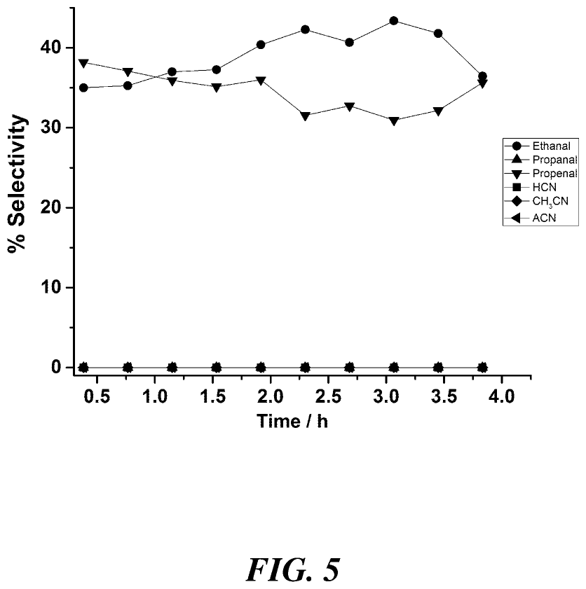

FIG. 5 depicts the selectivity vs time on stream plot of propene metathetic-oxidation under selected condition over Mo(═N-(2,6-C₆H₃-ⁱPr₂)₂(CH₂tBu)₂/Bi₂O₃—SiO₂₋₂₀₀ (feed ratio: C₃═/NH₃/O₂/N₂/He=7.5/0/10/12.5/70), according to one or more embodiments of the present disclosure.

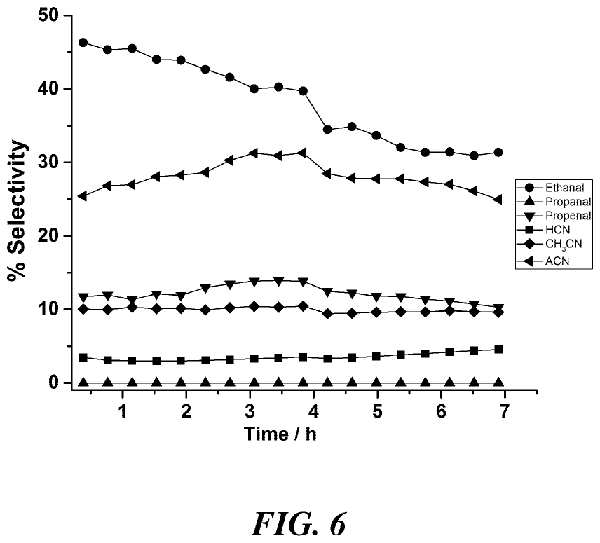

FIG. 6 depicts the selectivity vs time on stream plot of propene oxidation (FIG. 5) and ammoxidation (FIG. 6) under selected condition over Mo(═N-(2,6-C₆H₃—ⁱPr₂)₂(CH₂tBu)₂/Bi₂O₃—SiO₂₋₍₂₀₀₎ (feed ratio: C₃═/NH₃/O₂/N₂/He=7.5/7.5/2.5/12.5/70), according to one or more embodiments of the present disclosure.

### Example 15

The catalyst Mo(═N-t-Bu)₂(CH₂tBu)₂/SiO₂—Bi₂O₃₋₍₂₀₀₎ is prepared according to the method disclosed in detail in the above standard procedure II with a different support (Bismuth doped silica) (Bi=25% wt).

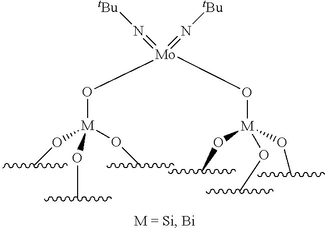

Table 5 gives the catalytic metathetic-oxidation and/or ammoxidation results obtained with Mo(═N-t-Bu)₂(CH₂tBu)₂/SiO₂—Bi₂O₃₋₍₂₀₀₎) catalyst.

TABLE 5

| | Feed ratio | % Conv | % Selectivity | | | | |
|---|---|---|---|---|---|---|---|
| Entry | C₃ = /NH₃/O₂/N₂/He | C₃ = | Ethanal | Propenal | HCN | CH₃CN | ACN |
| 18 | 7.5/0/10/12.5/70 | 3.5-5.5 | 22-29.5 | 44-52 | 0 | 0 | 0 |
| 19 | 7.5/1.875/7.5/12.5/70.6 | 3-6 | 25-29 | 5-6 | 3-4 | 18-20 | 30-34 |
| 20 | 7.5/1.875/10/12.5/68.1 | 3-5 | 28-30 | 6 | 3-4 | 17-19 | 31-34 |
| 21 | 7.5/3.75/10/12.5/66.3 | 5-6 | 28-29 | 3-4 | 3-4 | 18-19 | 34-38 |
| 22 | 7.5/7.5/2.5/12.5/70 | 4-5.5 | 13-17 | 0-3 | 6-6 | 31-32 | 44-45 |
| 23 | 7.5/7.5/10/12.5/62.5 | 4-6 | 8-9 | 2-2.5 | 3-4 | 25-27 | 47-51 |

Reaction conditions: T = 380° C.;
weight catalyst: 42 mg;
Total flow: 32 mL/min;
Gas hourly space velocity (GHSV) of 45714 h⁻¹;
W/F: 0.0787 g · s · mL⁻¹;
ACN = acrylonitrile.
Other minor products with selectivity <1-2% and over oxidation products CO₂ and CO are not presented in the table.

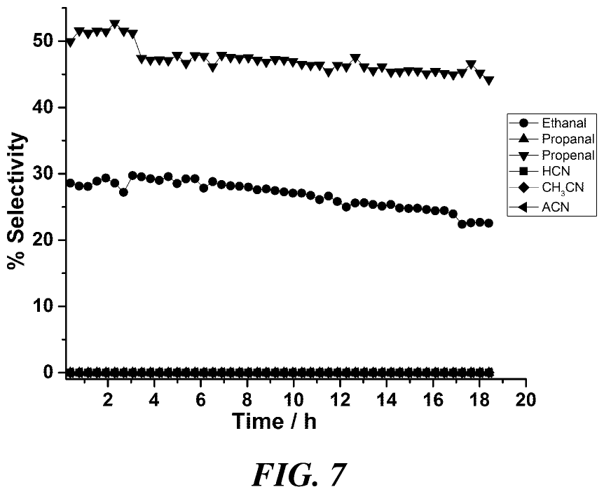

FIG. 7 depicts the selectivity vs time on stream plot of propene metathetic-oxidation under selected condition over Mo(═N-t-Bu)₂(CH₂tBu)₂/SiO₂—Bi₂O₃₋₍₂₀₀₎ (Feed ratio C₃═/NH₃/O₂/N₂/He=7.5/0/10/12.5/70), according to one or more embodiments of the present disclosure.

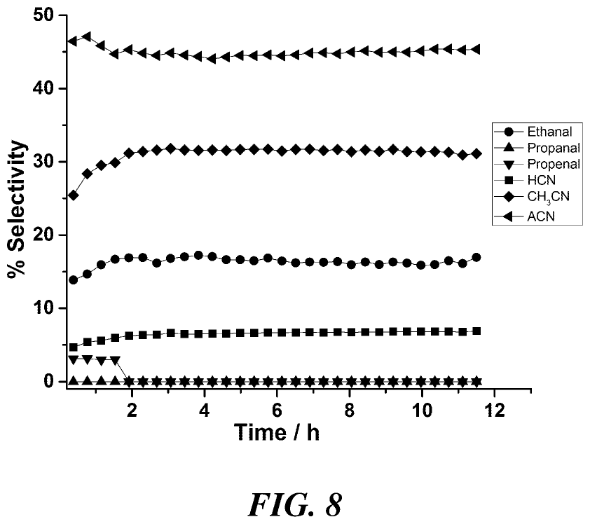

FIG. 8 depicts the selectivity vs time on stream plot of propene oxidation ammoxidation under selected condition over Mo(═N-t-Bu)₂(CH₂tBu)₂/SiO₂—Bi₂O₃₋₍₂₀₀₎ (Feed ratio: C₃═/NH₃/O₂/N₂/He=7.5/7.5/2.5/12.5/70), according to one or more embodiments of the present disclosure.

### Example 16

The catalyst Mo(═O)(CH₂^{t}Bu)₃C/SiO₂—Bi₂O₃₋₍₂₀₀₎ is prepared according to the method disclosed in detail in the above standard procedure IV with a different support (Bismuth doped silica).

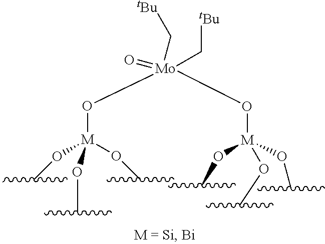

Table 6 gives the catalytic metathetic-oxidation and/or ammoxidation results obtained with Mo(═O)(CH₂^{t}Bu)₃C/SiO₂—Bi₂O₃₋₍₂₀₀₎ catalyst

TABLE 6

| | Feed ratio | % Conv | % Selectivity | | | | | |
|---|---|---|---|---|---|---|---|---|
| Entry | C₃ = /NH₃/O₂/N₂/He | C₃ = | Ethanal | Propanal | Propenal | HCN | CH₃CN | ACN |
| 24 | 7.5/0/5/12.5/75 | 3.5-5 | 30-36 | 5-6 | 41-57 | 0 | 0 | 0 |
| 25 | 7.5/0/10/12.5/70 | 2-4 | 34-40 | 5-8 | 36-45 | 0 | 0 | 0 |
| 26 | 7.5/7.5/2.5/12.5/70 | 3-4 | 26-38 | 0 | 7-11 | 4-5 | 18-24 | 21-38 |
| 27 | 7.5/7.5/10/12.5/62.5 | 3.5-5 | 29-30 | 0 | 7-8 | 4-5 | 20-21 | 26-27 |

Reaction conditions: T = 380° C.;
weight of catalyst: 36 mg;
Total flow: 32 mL/min;
Gas hourly space velocity (GHSV) of 53333 h⁻¹;
W/F: 0.067 g · s · mL⁻¹; ACN = acrylonitrile.
Other minor products with selectivity <1% and over oxidation products CO₂ and CO are not presented in the table.

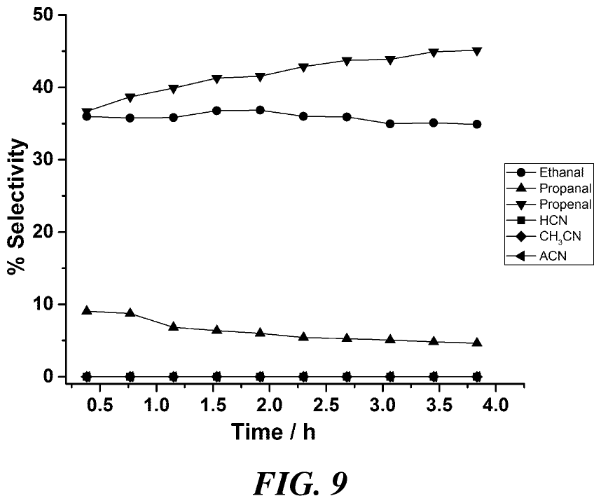

FIG. 9 depicts the selectivity vs time on stream plot of propene metathetic-oxidation under selected condition over Mo(═O)(CH₂^{t}Bu)₃C/SiO₂—Bi₂O₃₋₍₂₀₀₎ catalyst (Reaction conditions: T=380° C.; weight of catalyst: 36 mg; Feed ratio: C₃═/NH₃/O₂/N₂/He=7.5/0/10/12.5/70), according to one or more embodiments of the present disclosure.

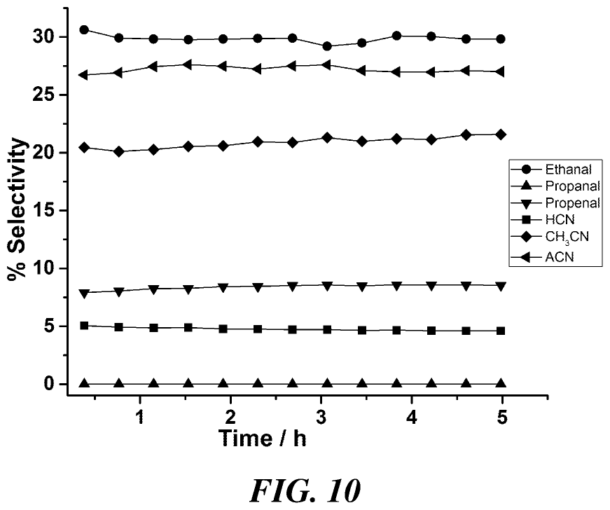

FIG. 10 depicts the selectivity vs time on stream plot of propene ammoxidation under selected condition over Mo(═O)(CH₂^{t}Bu)₃Cl/SiO₂—Bi₂O₃₋₍₂₀₀₎ catalyst (Feed ratio: C₃═/NH₃/O₂/N₂/He=7.5/7.5/10/12.5/62.5), according to one or more embodiments of the present disclosure.

### Example 17

The catalyst Mo(≡C^{t}Bu)(CH₂^{t}Bu)₃/Bi₂O₃—SiO₂₋₂₀₀ (Bi=˜10.0% wt, Mo=1.89% wt) is prepared according to the method disclosed in detail in the above standard procedure III with a different support (Bismuth doped silica).

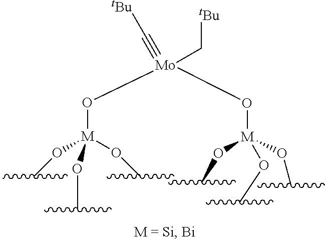

Table 7 depicts the catalytic metathetic-oxidation and/or ammoxidation results obtained with Mo(≡C^{t}Bu)(CH₂^{t}Bu)₃/Bi₂O₃—SiO₂₋₂₀₀

TABLE 7

| | Feed ratio | % Conv | Selectivity (%) | | | | |
|---|---|---|---|---|---|---|---|
| Entry | C₃ = /NH₃/O₂/N₂/He | C₃ = | Ethanal | Propenal | HCN | CH₃CN | ACN |
| 28 | 7.5/0/5/12.5/75 | 4-5 | 23-28 | 50-55 | 0 | 0 | 0 |
| 29 | 7.5/1.875/5/12.5/73.1 | 4-5 | 26-31 | 16-18 | 2.5-3 | 8-9 | 40-42 |
| 30 | 7.5/3.75/5/12.5/71.2 | 4-5.5 | 27 | 11-12 | 3 | 8-9 | 40-42 |
| 31 | 7.5/7.5/5/12.5/67.5 | 5-6 | 25-26 | 8 | 4 | 10-11 | 40-41 |

Reaction conditions: T = 380° C.;
weight catalyst: 30 mg;
Total flow: 32 mL/min;
Gas hourly space velocity (GHSV) of 64000 h⁻¹;
W/F: 0.056 g · s · mL⁻¹;
ACN = acrylonitrile.
Other minor products with selectivity <1-2% and over oxidation products CO₂ and CO are not presented in the table.

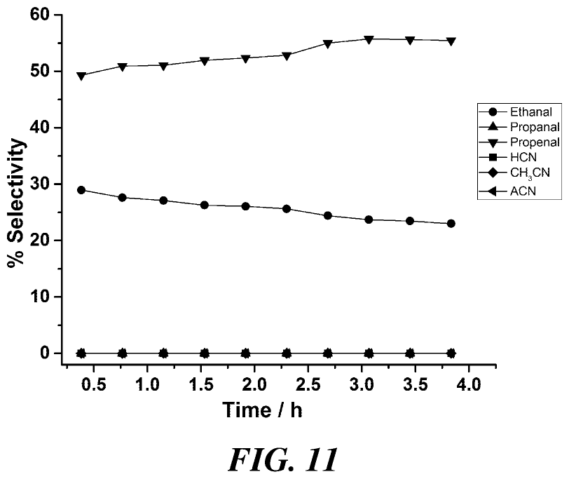

FIG. 11 depicts the selectivity vs time on stream plot of propene metathetic-oxidation over Mo(≡C^{t}Bu)(CH₂^{t}Bu)₃/Bi₂O₃—SiO₂₋₂₀₀ (Feed ratio: C₃═/NH₃/O₂/N₂/He=7.5/0/5/12.5/75), according to one or more embodiments of the present disclosure.

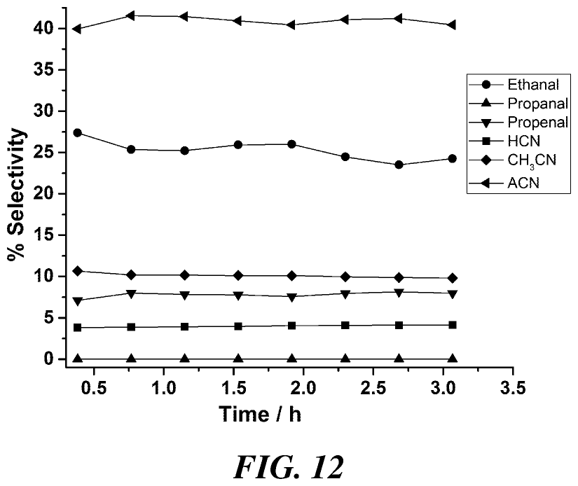

FIG. 12 depicts the selectivity vs time on stream plot of propene ammoxidation over Mo(≡C^{t}Bu)(CH₂^{t}Bu)₃/Bi₂O₃—SiO₂₋₂₀₀ (Feed ratio: C₃═/NH₃/O₂/N₂/He=7.5/7.5/5/12.5/67.5), according to one or more embodiments of the present disclosure.

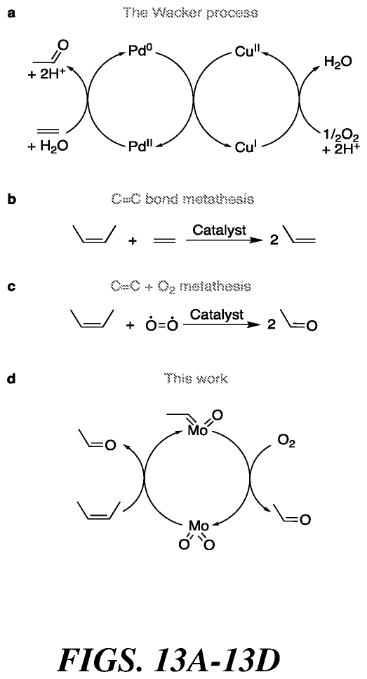

FIGS. 13A-13D show a comparison of the catalytic cycles for the oxidation of olefins to aldehydes and the reaction mechanisms: a) Bimetallic Wackercycle for the oxidation of ethylene to acetaldehyde; b) Cross metathesis of ethylene and 2-butene to propylene; c) Metathetic-oxidation of 2-butene and molecular oxygen to acetaldehyde; d) Cycle based on a silica-supported Mo (bis-oxo) single-atom species ((≡Si—O—)₂Mo(═O)₂) for the metathetic-oxidation of 2-butene to acetaldehyde, according to one or more embodiments of the present disclosure.

### Example 18

The supported single-site catalyst (≡Si—O—)₂Mo(═O)₂ is prepared according to the method disclosed in detail in the above standard procedure V with silica. This catalyst was tested for the metathetic-oxidation of terminal and internal olefins (propylene and 2-butene).

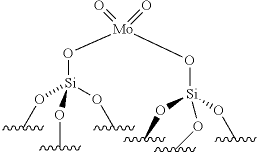

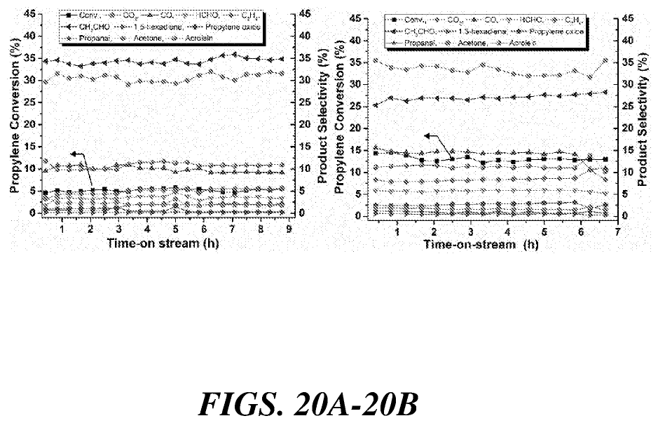

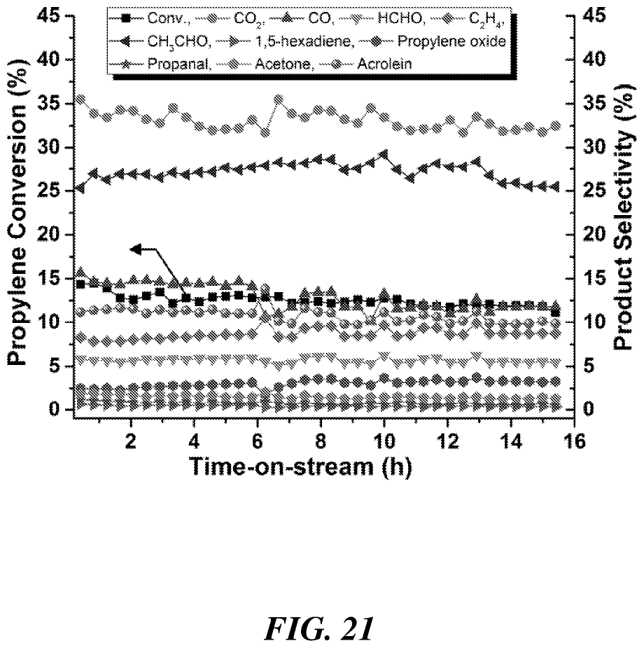

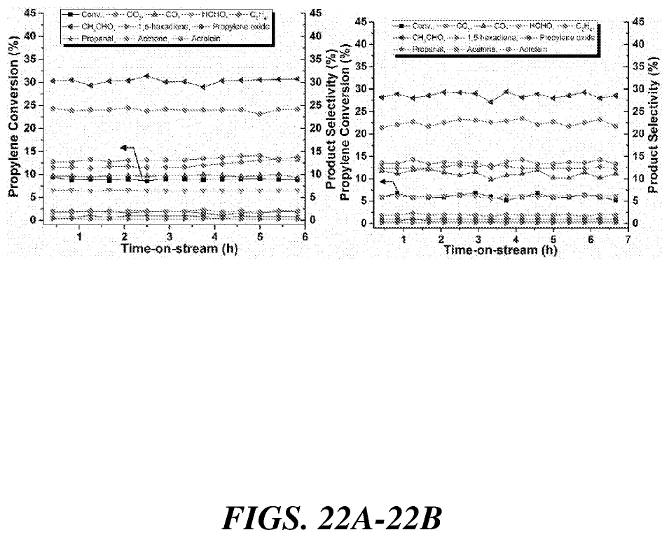

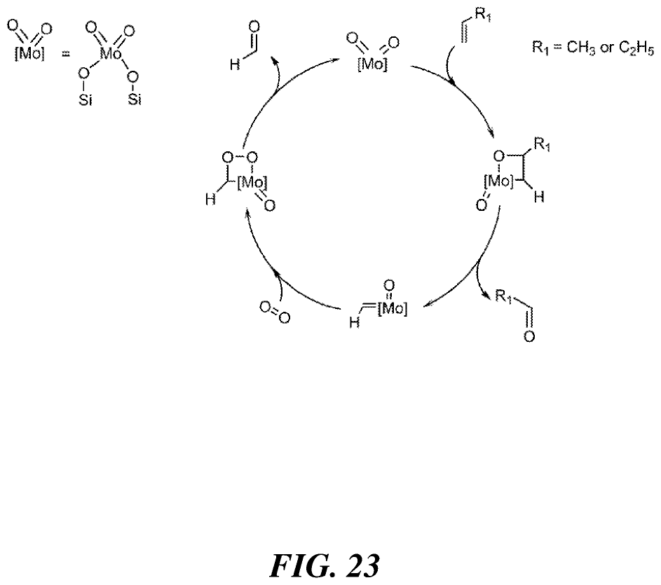

The initial studies, in which propylene was employed as the reactant under controlled oxidation conditions in a temperature range of about 400-450° C., exhibited unprecedented results. When propylene (7.5% v/v) in the presence of O₂ (10% v/v) came into contact with ((≡Si—O)₂Mo(═O)₂) at about 400° C. with only moderate to low propylene conversion (˜5-6%) was achieved. Nevertheless, the desired acetaldehyde oxidation product (about 30-35% selectivity) was produced along with CO and CO₂ (about 40-45% combined selectivity) and acrolein (˜10% selectivity) (Table 7a, Table 7b, and FIGS. 20A-20B). Other minor products including formaldehyde, acetone, ethylene, and propylene oxide were also observed. Increasing the reaction temperature to about 450° C. led to a much higher propylene conversion (˜12-14%) with only a slight change in the product selectivity. Importantly, the original activity and selectivity were preserved even after a prolonged experimental time of 16 h (FIG. 21), demonstrating that the catalyst was quite stable under the studied reaction conditions. The effect of the oxygen concentration in the feed on the propylene conversion and product selectivity was also investigated at about 450° C. (Table 7a, Table 7b, and FIGS. 22A-22B). At a lower oxygen to propylene ratio, the catalytic activity, especially the propylene conversion, markedly declined. Propylene conversions of ˜9% and ˜5.5% were observed when the feed gas mixture contained O₂:C₃H₆ ratios of 5:7.5 and 2.5:7.5, respectively. This observation was accompanied by a slightly higher formation of ethylene (approximately 12-13% vs 8-9% when O₂:C₃H₆ was 10:7.5), which may have been formed via a cracking pathway. As expected, the selectivity toward CO and CO₂ decreased. The formation of CO₂ and CO was primarily due to the thermal decomposition (decarboxylation and dehydration) of formic acid. In particular, CO was formed by the oxidation of formaldehyde, which was generated by [2+2] cycloaddition between α-olefins (propylene or 1-butene) and molecular oxygen (FIG. 23).

TABLE 7a
Summary of the catalytic oxidation results obtained with (≡SiO)₂Mo(=O)₂ using propylene as the reactant.

| Temp | Feed (%) | Conv | Selectivity (%) | | | |
|---|---|---|---|---|---|---|
| (° C.) | C₃₌, O₂, N₂, He | C₃ = (%) | CO₂ | CO | CH₃CHO | Acrolein |
| 400 | 7.5, 10, 12.5, 70 | 5-6 | 30-32 | 9-10 | 33-35 | 9-11 |
| 450 | 7.5, 10, 12.5, 70 | 12-14 | 31-34 | 11-14 | 25-29 | 9-12 |
| 450 | 7.5, 5, 12.5, 75 | 9-9.5 | 23-24 | 9-10 | 30-31 | 13-14 |
| 450 | 7.5, 2.5, 12.5, 77.5 | 5-6 | 21-23 | 10-11 | 28-29 | 10-11 |

TABLE 7b
Summary of the catalytic oxidation results obtained with ((≡SiO)₂Mo(=O)₂) using propylene as reactant.

| | | | Selectivity | | | | | | | | | |
|---|---|---|---|---|---|---|---|---|---|---|---|---|
| Temp | Feed (%) | Conv | | | | | | | | Propylene | | |
| (° C.) | C₃₌, O₂, N₂, He | C₃ = (%) | CO₂ | CO | Acetone | CH₃CHO | HCHO | Ethylene | Propanal | oxide | 1,5-hexadiene | Acrolein |
| 400 | 7.5, 10, 12.5, 70 | 5-6 | 30-32 | 9-10 | 2-3 | 33-35 | 3-4 | 4-6 | 0.5 | 1-2 | 0.2 | 9-11 |
| 450 | 7.5, 10, 12.5, 70 | 12-14 | 31-34 | 11-14 | 1-2 | 25-29 | 5-6 | 8-9 | 0.5-1 | 3 | 0.4 | 9-12 |
| 450 | 7.5, 5, 12.5, 75 | 9-9.5 | 23-24 | 9-10 | 2 | 30-31 | 6-6.5 | 11-13 | 0.5-1 | 1-2 | 0.4 | 13-14 |
| 450 | 7.5, 2.5, 12.5, 77.5 | 5-6 | 21-23 | 10-11 | 2 | 28-29 | 6 | 12.5 | 0.5 | 1 | 0.2 | 10-11 |

### Example 19

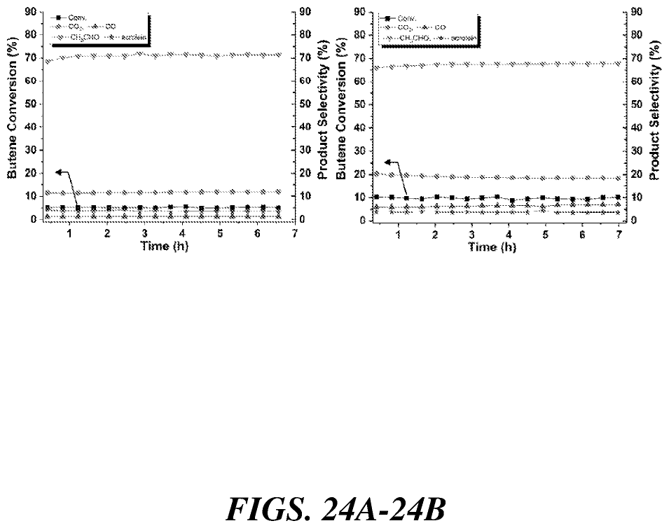

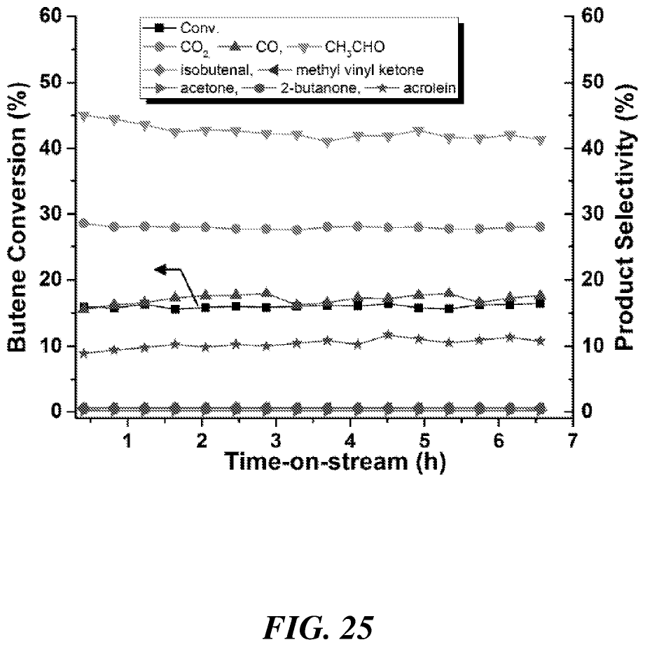

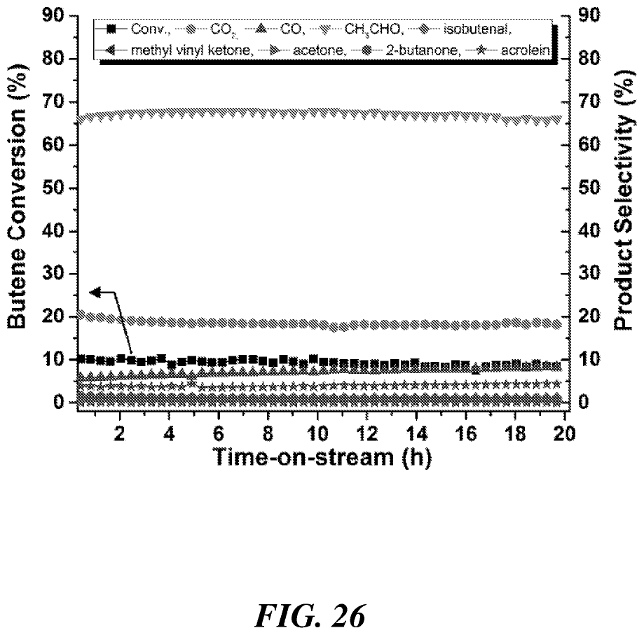

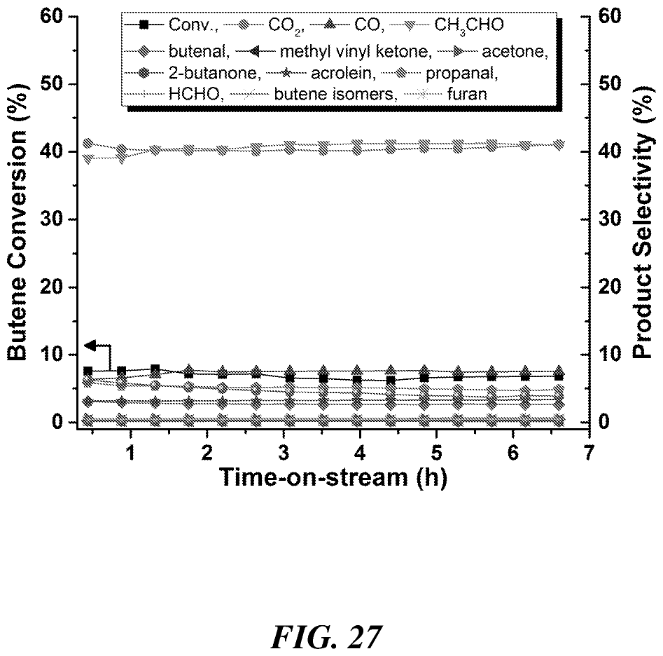

The unexpected catalytic results that were obtained with propylene inspired us to explore the scope of the oxidation reaction with internal olefins (i.e., cis-2-butene and cis-2-pentene). At about 400° C. in the presence of O₂, cis-2-butene exhibited a superior selectivity toward the formation of acetaldehyde (˜70%) as well as a reduction in the formation of CO and CO₂ (˜20-25% combined selectivity) (Table 8, Table 8a, and FIG. 24A-24B). Approximately 10% conversion of cis-2-butene was achieved at this temperature. A decrease in the temperature to about 350° C. resulted in a minor improvement in the acetaldehyde selectivity (˜71-72%) and a notable decrease in the olefin conversion (approximately 5%). An increase in the temperature to 450° C. resulted in a higher olefin conversion (˜15-16%) (FIG. 25), a significant drop in the acetaldehyde selectivity (˜41-44%), and a slight increase in the acrolein selectivity. As with propylene, the original activity using cis-2-butene as the substrate was preserved up to 20 h (FIG. 26), confirming the stability of the catalyst under the studied reaction conditions. When cis-2-pentene (5.9% v/v) was reacted at about 400° C. in the presence of O₂ (7.8% v/v) (FIG. 27 and Table 8a), moderate to good selective formation of acetaldehyde (41% selectivity), CO and CO2 (approximately 45% combined selectivity), and other minor products including acrolein, propanal, and methacrolein was observed.

TABLE 8a
Summary of the catalytic oxidation results obtained with ((≡SiO)₂Mo(=O)₂) using cis-2-butene as reactant

| Temp | Feed (%) | Conv | Selectivity (%) | | | |
|---|---|---|---|---|---|---|
| (° C.) | C₄₌, O₂, N₂, He | C₄₌ (%) | CO₂ | CO | CH₃CHO | Acrolein |
| 350 | 7.5, 10, 12.5, 70 | 5 | 12-14 | 2-3 | 71 | 3-4 |
| 400 | 7.5, 10, 12.5, 70 | 9-10 | 17-20 | 6-8 | 66-68 | 3-4 |
| 450 | 7.5, 10, 12.5, 70 | 15-16 | 27-28 | 15-17 | 41-44 | 10-11 |

TABLE 8b
Summary of the catalytic oxidation results obtained with ((≡SiO)₂Mo(=O)₂) using cis-2-butene as reactant

| | | | Selectivity | | | | | | | |
|---|---|---|---|---|---|---|---|---|---|---|
| Temp | Feed (%) | Conv | | | | | | Methyl | | |
| (° C.) | C₄₌, O₂, N₂, He | C₄ = (%) | CO₂ | CO | Acetone | CH₃CHO | Isobutenal | vinyl ketone | 2-butanone | Acrolein |
| 350 | 7.5, 10, 12.5, 70 | 5 | 12-14 | 2-3 | 0.5 | 71 | 0.5 | 2 | 2-3 | 3-4 |
| 400 | 7.5, 10, 12.5, 70 | 9-10 | 17-20 | 6-8 | 0.3 | 66-68 | 0.4 | 1 | 1-1.5 | 3-4 |
| 450 | 7.5, 10, 12.5, 70 | 15-16 | 27-28 | 15-17 | 0.15 | 41-44 | 0.6 | 0.6 | 0.5-1 | 10-11 |

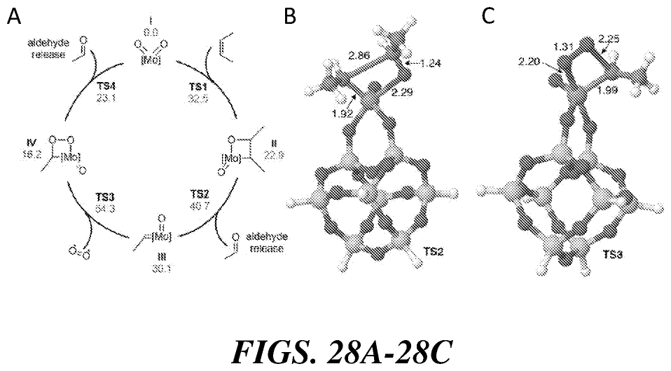

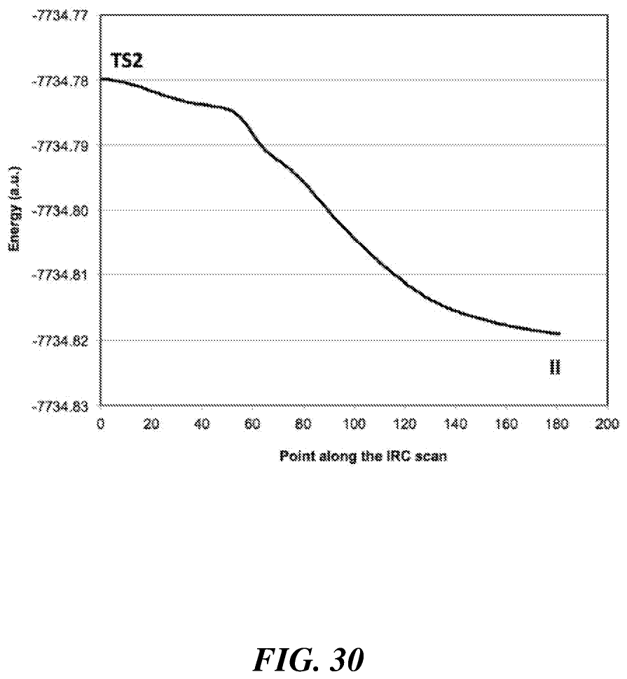

Computational studies. Density functional theory (DFT) calculations were performed to obtain a more complete understanding of the reaction pathway. The energetics of all the transformations for the oxidation of cis-2-butene are reported in FIG. 28A-28C, see also FIG. 30. The reaction started with the conversion of the initial silica-supported Mo(VI) bis-oxo species (I in FIG. 28A-28C) into the metallacyclobutane-like intermediate (II) via [2+2] cycloaddition of 2-butene with one of the W═O bonds. This step was endergonic by about 22.9 kcal/mol and required about 32.5 kcal/mol of Gibbs free energy of activation to move through transition state TS1. An intermediate that corresponded to the coordination of cis-2-butene to I did not play a role in the reaction kinetics because it was less stable by about 4.4 kcal/mol than the infinitely separated cis-2-butene and I. Cyclo-elimination from II via transition state TS2 at about 40.7 kcal/mol liberated an acetaldehyde molecule and led to Mo-oxocarbene III. This step was endergonic by about 7.2 kcal/mol.

The catalytic cycle was completed by reaction of III with molecular oxygen. The transformation occurred through transition state TS3 and involved a moderate activation barrier of about 24.2 kcal/mol. The reactants (i.e., III and molecular oxygen) were in the singlet and triplet spin states, respectively. Therefore, TS3 had a triplet spin state. Attempts to locate TS3 with a singlet spin state failed because the geometry optimizations always led to metallacycle intermediate IV. Further, the energy of TS3 in the singlet spin state using the triplet spin state geometry was about 22.4 kcal/mol higher than in the triplet spin state, supporting the hypothesis that the [2+2] cycloaddition between III and O₂ occurred via a transition state in a triplet spin state. Metallacycle IV in the singlet spin state was located about 13.9 kcal/mol below III+O₂. The energy of IV in the triplet spin state using the singlet spin state geometry was about 34.1 kcal/mol higher than in the singlet spin state, suggesting that spin state flipping occurred during the relaxation of TS3 to IV. Cyclo-elimination of acetaldehyde from singlet IV regenerated I in the singlet spin state via transition state TS4 and a low energy barrier of about 6.9 kcal/mol. This process closed the catalytic cycle.

Overall, the oxidation of cis-2-butene to two acetaldehyde molecules was strongly exergonic with a Gibbs free energy change of about −84.2 kcal/mol. Because the reaction was performed in a flow reactor, the kinetics of the two metathesis events were considered separately, and no equilibrium condition among the reactants, products, and intermediates can be established. The first metathesis event from I to III had an overall energy change of about 40.7 kcal/mol from I+cis-2-butene to the cyclo-elimination transition state, TS2 (FIG. 28B). This energy change corresponded to a reaction half-time of ˜6 seconds at about 350° C., which was consistent with the experimental conditions. The second metathesis event from III to I had an overall energy change of about 24.2 kcal/mol from III+O₂ to the [2+2] cycloaddition transition state TS3 (FIG. 28C). Thus, the energy change of the first metathesis event was lower than that of the second metathesis event, making the former event the rate determining step.

The catalytic results confirmed the starting hypothesis that high temperatures can promote metathetic oxidation of olefins by molecular oxygen using (≡Si—O—)₂Mo(═O)₂. In this study, cis-2-butene was oxidized by O₂ to acetaldehyde with a selectivity higher than about 70% at about 10% conversion using a silica-supported single-site catalyst (≡Si—O—)₂Mo(═O)₂. Our DFT calculations indicated that the reaction occurred via “metathetic” oxidation with the formation of metallacycle intermediates.

Using a Mo bis-oxo species to selectively cleave an olefinic double bond to yield the corresponding aldehyde as the product was a new reaction in the field of oxidation. No previously reported single-site catalytic system has been capable of promoting the direct oxidation of propylene or cis-2-butene to acetaldehyde via O₂. Therefore, the results introduced new perspectives for organic synthesis and green chemistry. Metathetic oxidation is a simple approach for producing aldehydes that avoid the expensive bimetallic system of the Wacker process or the tedious steps of hydroformylation, which are both large-scale industrial processes.

### Example 20

At about 400° C. in the presence of O₂, cis-2-pentene exhibited a selectivity toward the formation of acetaldehyde (˜40%) (Table 9).

TABLE 9
Summary of the catalytic oxidation results obtained with [(≡SiO)₂Mo(=O)₂] using cis-2-pentene as reactant

| | | | Selectivity (%) | | | | | | | | | | |
|---|---|---|---|---|---|---|---|---|---|---|---|---|---|
| | Feed (%) | | | | | | | Methyl | 2- | | | | |
| Temp. | C₃₌, O₂, | Conv | | | | | | vinyl | butanone/ | | | | |
| (° C.) | N₂, He | C₃₌ (%) | CO₂ | CO | Acetone | CH₃CHO | Isobutenal | ketone | HCHO | Butenes | Furan | Acrolein | Propanal |
| 400 | 5.9, 7.8, | 7 | 40 | 7 | 0.5 | 40-41 | 2.6 | 0.3 | 0.2/0.6 | 0.2 | 0.4 | 3 | 5 |
| | 12.5, 73.8 | | | | | | | | | | | | |

Other embodiments of the present disclosure are possible. Although the description above contains much specificity, these should not be construed as limiting the scope of the disclosure, but as merely providing illustrations of some of the presently preferred embodiments of this disclosure. It is also contemplated that various combinations or sub-combinations of the specific features and aspects of the embodiments may be made and still fall within the scope of this disclosure. It should be understood that various features and aspects of the disclosed embodiments can be combined with or substituted for one another in order to form various embodiments. Thus, it is intended that the scope of at least some of the present disclosure should not be limited by the particular disclosed embodiments described above.

Thus the scope of this disclosure should be determined by the appended claims and their legal equivalents. Therefore, it will be appreciated that the scope of the present disclosure fully encompasses other embodiments which may become obvious to those skilled in the art, and that the scope of the present disclosure is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims.

The foregoing description of various preferred embodiments of the disclosure have been presented for purposes of illustration and description. It is not intended to be exhaustive or to limit the disclosure to the precise embodiments, and obviously many modifications and variations are possible in light of the above teaching. The example embodiments, as described above, were chosen and described in order to best explain the principles of the disclosure and its practical application to thereby enable others skilled in the art to best utilize the disclosure in various embodiments and with various modifications as are suited to the particular use contemplated. It is intended that the scope of the disclosure be defined by the claims appended hereto

Various examples have been described. These and other examples are within the scope of the following claims.

1. A catalyst, comprising:
a support including one or more of inorganic oxide, silicon-modified inorganic oxide, and bismuth-modified inorganic oxide; and an inorganic and/or organometallic complex grafted on the support; wherein the complex includes one or more of Group V elements, Group VI elements, and Group VII elements.

2. The catalyst of claim 1, wherein the support includes one or more of silica, bismuth oxide, bismuth-modified silica, and silicon-modified bismuth oxide.

3. The catalyst of claim 1, wherein the complex includes one or more of Mo, W, Cr, and Re.

4. The catalyst of claim 1, wherein an active metal content of the catalyst is less than about 20 wt %.

5. The catalyst of claim 1, wherein the catalyst is a single-site catalyst.

6. The catalyst of claim 1, wherein the catalyst is a bipodal tetrahedral monometallic and/or bimetallic catalyst characterized by one or more of the following:
a. A bipodal tetrahedral catalyst and/or precatalyst having a metallic site which is four coordinated in which two bonds are anchored to the support through an oxygen atom and represented by
i. either the formula 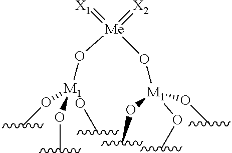
wherein X₁ and X₂ are the same or different and are selected from O, NH, and/or NR, wherein R is selected from alkyl, aryl, tris-alkylsilyl, tris-arylsilyl, tris-alkylstanyl, and/or tris-arylstanyl;
ii. and/or by the formula 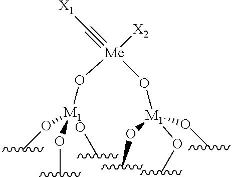
wherein X₁ and X₂ are the same or different and wherein X₁ is selected from alkyl, aryl, trimethylsilyl, and/or H, and X₂ is selected from alkyl, aryl, alkoxy, aryloxy, thio-aryloxy, and/or amide;
iii. and/or by the formula 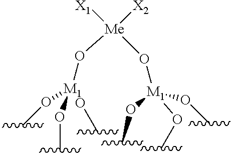
wherein X₁ and X₂ are the same or different and are selected from allyl and/or substituted allyl and/or aryl and/or alkoxy and/or aryloxy and/or amide;
iv. and/or by the formula 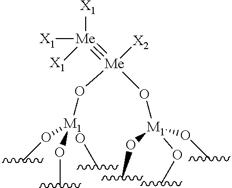
wherein X₁ and X₂ are the same or different and are selected from alkyl, aryl, alkoxy, aryloxy thio-aryloxy, and/or amide;
and wherein “M1” denote silicon and/or bismuth which are part of the oxide support, and “Me” denotes a metal of group VI.

7. The catalyst of claim 1, wherein the catalyst is a bipodal pentahedral monometallic and/or bimetallic catalyst characterized by one or more of the following:
a. a bipodal pentahedral catalyst and/or precatalyst with a metallic site which is penta-coordinated and in which two bonds are anchored to the support through an oxygen atom and represented by the formula 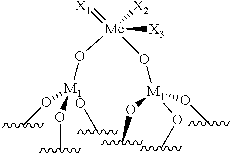
wherein X₁ is selected from O, and/or NH, and/or NR and/or CHR, wherein R is selected from alkyl and/or aryl wherein X₂ and X₃ are the same or different and are selected from alkyl, and/or aryl, and/or alkoxy and/or aryloxy and/or siloxy and/or thio-aryloxy and/or amide and/or pyrolidyl and/or substituted pyrolidyl; and
b. a bipodal pentahedral dimeric catalysts and/or precatalysts with a metallic site which is penta-coordinated and in which two bonds are anchored to the support through an oxygen atom and represented by the formula 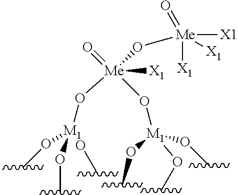
wherein X₁ is selected from alkyl and/or aryl and/or alkoxy and/or aryloxy and/or thioaryloxy and/or siloxy and/or thio-aryloxy and/or amide and/or pyrolidyl and/or substituted pyrolidyl;
and wherein “M1” denote silicon and/or bismuth which are part of the oxide support, and “Me” denotes a metal of group VI elements.

8. The catalyst of claim 1, wherein the catalyst is a bipodal hexahedral monometallic catalyst characterized by one or more of the following chemical formulas:
a. a bipodal hexahedral catalysts and/or precatalysts with a metallic site which is hexa-coordinated and in which two bonds are anchored to the support through an oxygen atom and represented by the formula 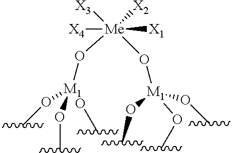
wherein X₁, X₂, X₃, and X₄ are the same or different and are selected from alkyl and/or aryl and/or alkoxy and/or aryloxy and/or thioaryloxy and/or siloxy and/or amide;
and wherein “M1” denote silicon and/or bismuth which are part of the oxide support, and “Me” denotes a metal of group VI elements.

9. The catalyst of claim 1, wherein the catalyst is a monopodal tetrahedral monometallic and/or bimetallic catalyst characterized by one or more of the following chemical formulas:
a. a monopodal tetrahedral catalyst and/or precatalyst wherein the metallic site is four coordinated and in which one bond is anchored to the support through an oxygen atom and represented by
i. either the formula 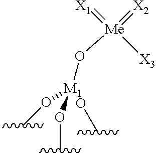
wherein X₁ and X₂ are the same or different and are selected from O, NH, and/or NR wherein R is selected from alkyl and/or aryl, and X₃ is selected from alkyl, and/or aryl and/or alkoxy and/or aryloxy and/or thioaryloxy and/or siloxy and/or amide;
ii. and/or the formula 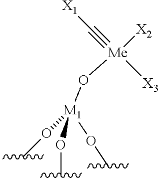
wherein X₁ is selected from alkyl, aryl, and/or H, and X₂ and X₃ are the same or different and are selected from alkyl, aryl, alkoxy, aryloxy, thio-aryloxy, siloxy, and/or amide;
iii. and/or the formula 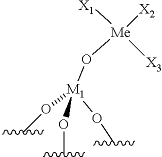
wherein X₁, X₂ and X₃ are the same or different and are selected from allyl and/or substituted allyl and/or aryl and/or alkoxy and/or aryloxy and/or thioaryloxy and/or siloxy and/or amide;
iv. and/or the formula 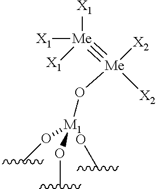
wherein X₁ and X₂ are the same or different and are selected from allyl and/or substituted allyl and/or aryl and/or alkoxy and/or aryloxy and/or thioaryloxy and/or amide;
and wherein “M1” denote silicon and/or bismuth which are part of the oxide support, and “Me” denotes a metal of group VI elements.

10. The catalyst of claim 1, wherein the catalyst is a monopodal pentahedral monometallic and/or bimetallic catalyst characterized by one or more of the following chemical formulas:
a. a monopodal pentahedral catalyst and/or precatalyst wherein the metallic site is penta-coordinated in which one bonds is anchored to the support through an oxygen atom and represented by 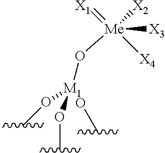
wherein X₁ is selected from O, NH, NR, and/or CHR, wherein R is selected from alkyl and/or aryl, and wherein X₂, X₃ and X₄ are the same or different and are selected from alkyl and/or aryl and/or alkoxy and/or aryloxy and/or thioaryloxy and/or siloxy and/or thio-aryloxy and/or amide and/or pyrolidyl and/or substituted pyrolidyl; or
b. a monopodal pentahedral dimeric catalyst and/or precatalyst wherein the metallic site is penta-coordinated and in which one bond is anchored to the support through an oxygen atom and represented by 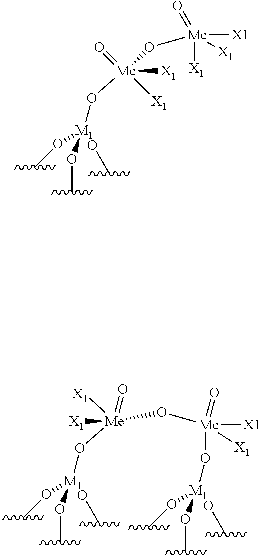
wherein X₁ is selected from alkyl and/or aryl and/or alkoxy and/or aryloxy and/or siloxy and/or thio-aryloxy and/or amide and/or pyrolidyl and/or substituted pyrolidyl; or
and wherein “M1” denote silicon and/or bismuth which are part of the oxide support, and “Me” denotes a metal of group VI elements.

11. The catalyst of claim 1, wherein the catalyst is a monopodal hexahedral monometallic and/or bimetallic catalyst characterized by one or more of the following chemical formulas:
a. a monopodal hexahedral catalyst and/or precatalyst wherein the metallic site is hexa-coordinated and in which two bonds are anchored to the support through an atom of oxygen and represented by 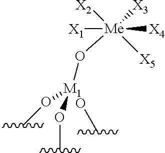
wherein X₁, X₂, X₃, X₄ and X₅ are the same or different and are selected from R wherein R is selected from alkyl and/or aryl and/or alkoxy and/or aryloxy and/or siloxy and/or thio-aryloxy, and/or amide;
and wherein “M1” denote silicon and/or bismuth which are part of the oxide support, and “Me” denotes a metal of group VI elements.

12. The catalyst of claim 1, wherein the catalyst is characterized by one or more of the following chemical formulas: 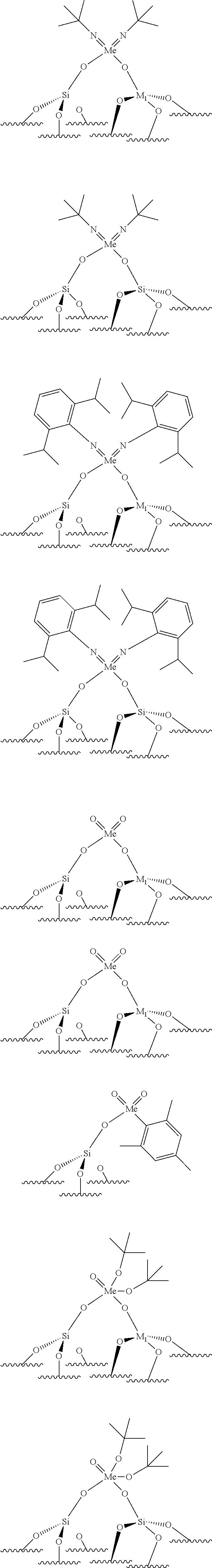 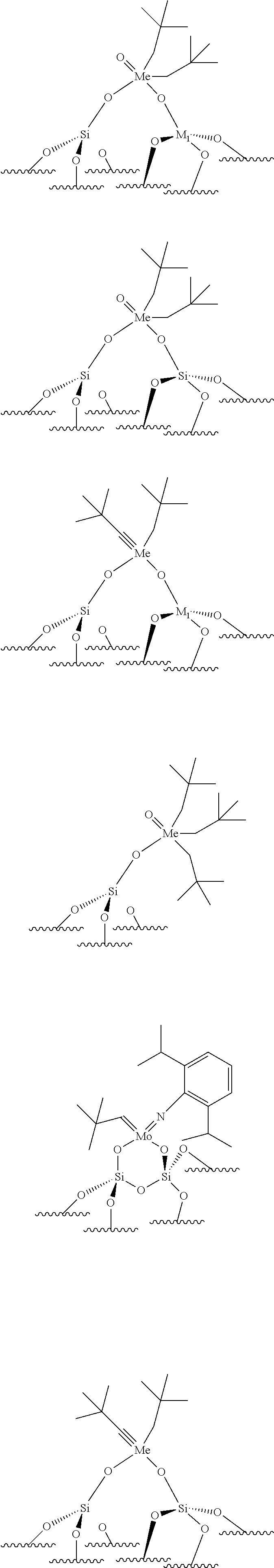
where Si is part of the oxide support and includes one or more of silicon and bismuth, M₁ is part of the oxide support and includes one or more of silicon and bismuth, where Me is one or more of Group VI elements.

13. A method of making one or more of aldehydes and nitriles, comprising:
contacting an olefin and one or more of oxygen and ammonia in a presence of a catalyst to produce one or more of aldehydes and nitriles; wherein the catalyst is a single-site catalyst including an inorganic and/or organometallic complex grafted on a support.

14. The method of claim 13, wherein the contacting proceeds at a temperature ranging from about 350° C. to about 450° C.

15. The method of claim 13, wherein the olefin includes one or more of a terminal olefin and internal olefin.

16. The method of claim 13, wherein the olefin includes hydrocarbons with 2 to 5 carbons.

17. The method of claim 13, wherein the olefin includes one or more of propylene, isobutene, cis-2-butene, 1-butene, and cis-2-pentene.

18. The method of claim 13, wherein the aldehydes include one or more of ethanal, propanal, and propenal.

19. The method of claim 13, wherein the nitriles include one or more of acrylonitrile and acetonitrile.

20. A method of making a catalyst, comprising:
treating one or more of an inorganic oxide support, silicon-modified inorganic oxide support, and bismuth-modified inorganic oxide support at or to a temperature ranging from about 100° C. to about 900° C.; and grafting one or more of an inorganic and/or organometallic complex on the support.
